SEQUENCE LISTING (23 sequences)

SEQ ID NO: 1 - protein, 210 aa, Borrelia burgdorferi
MKKNTLSAILMTLFLFISCNNSGKDGNASANSADESVKGPNLTEISKKITESNAVVLAVKEVETLLASINQLAKAIGKKIDQNGTLGDDGGQNGSLLAGAYAISTVIIEKLSTLKNVEELKEKITKAKDCSEKFAGKLKNEHASLGKKDATDDDAKKAILKTHGNTDKGAKELKDLSDSVESLVKAAKEMLTNSVKELTSPVVAESPKKP

SEQ ID NO: 2 - protein, 209 aa, Borrelia valaisiana
MKKNTLSAILMTLFLFISCNNSGGDTASTNPVDESAKGPNLTEISKKITDSNAIVLAVKEVETLLASINEIANKGIGKKINQNGLDNLTDHNGSLIAGAYVISTLITEKLNNLKNSEGLKEKIKKVKECSDKFTKKLTTSNGDLGKENVTDAHAQAAILKTNPTNDKGAKELGELFESVEILSKAAQEALTNSIAELTSPVVAENPKNP

SEQ ID NO: 3 - protein, 208 aa, Borrelia garinii
MKKNTLSAILMTLFLFISCNNSGGDTASTNPDESAKGPDLTVISKKITDSNAFVLAVKEVEALISSIDELANKAIGKVIHQNNGLNANAGQNGSLLAGAYAISTLITEKLSKLKNSEENKKIEEAKNHSEAFTNRLKGSHAQLGVAAATDDHAKEAILKSNPTKDKGAKELKDLSESVESLAKAAQEALANSVKELTNPVVAETPKKL

SEQ ID NO: 4 - protein, 211 aa, Borrelia afzelii
MKKNTLSAILMTLFLFISCNNSGKGGDSASTNPADESAKGPNLTEISKKITDSNAFVLAVKEVETLVSSIDELANKAIGKKIQQNGLGAEANRNESLLAGVHEISTLITEKLSKLKNSGELKAKIEDAKKCSEEFTNKLRVSHADLGKQGVNDDDAKKAILKTNADKTKGAEELGKLFKSVEGLVKAAQEALTNSVKELTSPVVAESPKKP

SEQ ID NO: 5 - protein, 700 aa, Borrelia burgdorferi
MKKMLLIFSFFLIFLNGFPVSAREVDREKLKDFVNMDLEFVNYKGPYDSTNTYEQIVGIGEFLARPLTNSNSNSSYYGKYFINRFIDDQDKKASVDVFSIGSKSELDSILNLRRILTGYLIKSFDYDRSSAELIAKVITIYNAVYRGDLDYYKGFYIEAALKSLSKENAGLSRVYSQWAGKTQIFIPLKKDILSGNIESDIDIDSLVTDKVVAALLSENEAGVNFARDITDIQGETHKADQDKIDIELDNIHESDSNITETIENLRDQLEKATDEEHKKEIESQVDAKKKQKEELDKKAINLDKAQQKLDSAEDNLDVQRNTVREKIQEDINEINKEKNLPKPGDVSSPKVDKQLQIKESLEDLQEQLKETGDENQKREIEKQIEIKKSDEKLLKSKDDKASKDGKALDLDRELNSKASSKEKSKAKEEEITKSKSQKSLGDLNNDENLMMPEDQKLPEVKKLDSKKEFKPVSEVEKLDKIFKSNNNVGELSPLDKSSYKDIDSKEETVNKDVNLQKTKPQVKDQVTSLNEDLTTMSIDSSSPVFLEVIDPITNLGTLQLIDLNTGVRLKESTQQGIQRYGIYEREKDLVVIKMDSGKAKLQILDKLENLKVVSESNFEINKNSSLYVDSKMILVAVRDKDSSNDWRLAKFSPKNLDEFILSENKIMPFTSFSVRKNFIYLQDEFKSLVILDVNTLKKVK

SEQ ID NO: 6 - protein, 369 aa, Borrelia burgdorferi
MKKITSLVFILPLFVLITCKNTVSDEVIKNKLLNSIETLGKEFSDAFISSPFGGSLGTAAIDLKKSEVRNYFETISKGFKKIQEGFKKISFDFAEYDNAVHSINDIAKVFDWYLMEFVNDVEEAAKAADIDSDLVGQVVAVNPKIADSSSVKRFANWIQTLLRFSSVFGYDIYNKMFKNINIDSSNHRKHMQQVGNVKHVGKIFLKKENEDNHNCGTNNGSAENKAKDLVNSVSGEEILCLILRDIGGSPEHAKGKSPQSSENDPIEAAIGTNYAGDANSSSNCFVYDMPVNIAAAIVLRGMAKDGQFFAINGENDTVKTAVEIGVTKTLVALTTLLRGSVGEMIGRLDQIRKERLRFGTLVENNGYRY

SEQ ID NO: 7 - protein, 397 aa, Borrelia garinii
MKKISSAIFTIVFLVFINCKSDARKAINSIQTQKFTSFDGLLIDGFLSLKPNPKKSEVKDYFNSMAKTLNKTKDKLAKLISEKGGKTTEENNTDTAKEDNSTVNPVDNEINKIKDMIDKMIDAANTIVETVAETATETMGEVVEVKSSGNVATKADVKSVVEIAKGIKKIVGAAGIADKLKAEADKTTKPISEESNNKEAGKMFSGKQGDQGGKVIDSDVISPEIGGGANPMDINKAAEAVKNVSGEQILGAIIAAAKAIESGGKATTEGKNADEAKNPIEAAIGGNDDSNATAFTGNMEKDTQIAAAIVLRGMAKNGKFAVKMGRGPSADGNTIRALVKNAANKTVDALSQLVLKAINESLTKIAKTIKAGGEAANEAVNSNFPSVKFAEKHLEDK

SEQ ID NO: 8 - protein, 187 aa, Borrelia burgdorferi
MIKCNNKTFNNLLKLTILVNLLISCGLTGATKIKLESSAKAIVDEIDAIKKKAASMGVNFDAFKDKKTGSGVSENPFILEAKVRATTVAEKFVIAIEEEATKLKETGSSGEFSAMYDLMFEVSKPLQELGIQEMTKTVSMAAEENPPTTAQGVLEIAKKMREKLQRVHKKNQDTLKKKNTEDSTAKS

SEQ ID NO: 9 - protein, 175 aa, Borrelia garinii
MTKYIKNLLKLTLIVGLLVACSLTGKARLESSVKDITDEIDKAIKEAIADGVKLNELEENKTGAKKGGPQIRDAKIRVINLSVKFLKEIEEEANILKDNVGMNKVDKDQLLKDMYDLMLNAAGSLQKLGLQEMIKTVTQAAEKTPPTTVEGILMIANTIEDKLKKIKGKQETNKK

SEQ ID NO: 10 - protein, 170 aa, Borrelia afzelii
MIKYNKIILTLTLLASLLAACSLTGKARLESSVKDITNEIEKAIKEAEDAGVKTDAFTETQTGGKVGGSQIRAAKIRVADLTIKFLEATEEETITFKENGAGEEDFSGIYDLILNAAKAVEKIGMQGMKQAVEEAAKEKPKTTADGIIAIVKVMKAKVENIKEKQTKNQK

SEQ ID NO: 11 - protein, 187 aa, Borrelia burgdorferi
MKIGKLNSIVIALFFKLLVACSIGLVERTNAALESSSKDLKNKILKIKKEATGKGVLFEAFTGLKTGSKVTSGGLALREAKVQAIVETGKFLKIIEEEALKLKETGNSGQFLAMFDLMLEVVESLEDVGIIGLKARVLEESKNNPINTAERLLAAKAQIENQLKVVKEKQNIENGGEKKNNKSKKKK

SEQ ID NO: 12 - protein, 184 aa, Borrelia garinii
MKKFNLIIVALFVALLAACNFGLTGEVKAMLESSSDNVKNKILQIKEEAAKKGVNFKAFTGTATGSKVANGGSALREAKVQAINEVEKFLNTIEKEALILKKNGNSSQFLAMFDLMLEVTGSLDEIGIKGIKSSISEEAKSNPVNTAERLVEVKAKIENKLEGVKKRQKLDDEEKKISKSKKNK

SEQ ID NO: 13 - protein, 183 aa, Borrelia afzelii
MKKFNLIIEALFAILLTACNFGLMEETKIALESSSKDVKNKILQIKKDAEDKGVNFAAFTSSETGSKVTNGGLALREAKIQAINEVEKFLKRIEEEALKLKEHGNSGQFLELFDLLLEVLESLEPIGIKGLKDFISEEAKCNPISTSERLIEVKVQIENKMEEVKRKQNLNKERKSNKGKKKK

SEQ ID NO: 14 - protein, 178 aa, Borrelia burgdorferi
MEKYLSYIKKDDLDAIQLKLQELLASLHIFYSNLRGIHWNIKDTNFFVIHKKTQKLYEYIEKIIDIVAERSRMLGYDSEFRYSEFMKKSFIKELDIESTSNFLPSMESIVCSLTEILKNIFGMRKLIDTAGDYGTANIMDDIMSDLEKHLWMHKALLENCDCFCHDENESKCCECDAK

SEQ ID NO: 15 - protein, 176 aa, Borrelia afzelii
MEKYLSYIKKDDLEEIHSKLQELLASLHIFYSNLRGIHWNIKDTNFFVIHKKTQDLYEYIEKIIDIIAERSRMLGYDSEFRYSEFMKKSFIKELNIESTSNFSLSMQSIVSSLAEILKNIFGMRKLIDTACDYGTANLIDDIMSDLEKYLWMHKALLENCDCVCHDDNKCCECDIK

SEQ ID NO: 16 - protein, 177 aa, Borrelia burgdorferi
DGKYDLIATVDKLELKGTSDKNNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGYVLEGTLTAEKTTLVVKEGTVTLSKNISKSGEVSVELNDTDSSAATKKTAAWNSGTSTLTI

SEQ ID NO: 17 - protein, 274 aa, Borrelia garinii
MKKYLLGIGLILALIACKQNVSSLDEKNSVSVDLPGGMKVLVSKEKDKDGKYSLMATVEKLELKGTSDKSNGSGVLEGEKADKSKAKLTISQDLNQTTFEIFKEDGKTLVSRKVNSKDKSSTEEKFNDKGKLSEKVVTRANGTRLEYTEIKNDGSGKAKEVLKGFALEGTLTDGGETKLTVTEGTVTLSKNISKSGEITVALNDTETTPADKKTGEWKSDTSTLTISKNSQKPKQLVFTKENTITVQNYNRAGNALEGSPAEIKDLAELKAALK

SEQ ID NO: 18 - protein, 130 aa, Borrelia afzelii
QNVSSLDEKNSASVDLPGEMKVLVSKEKDKDGKYSLKATVDKIELKGTSDKDNGSGVLEGTKDDKSKAKLTIADDLSKTTFELFKEDGKTLVSRKVSSKDKTSTDEMFNEKGELSAKTMTRENGTKLEYT

SEQ ID NO: 19 - protein, 336 aa, Borrelia burgdorferi
MIINHNTSAINASRNNAINAANLSKTQEKLSSGYRINRASDDAAGMGVSGKINAQITGLSQASRNTSKAINFIQTTEGNLNEVEKVLVRMKELAVQSGNGTYSDADRGSIQIEIEQLTDEINRIADQAQYNQMHMLSNKSASQNVKTAEELGMQPAKINTPASLSGSQASWTLRVHVGANQDEAIAVNIYSANVANLFAGEGAQAAQAAPVQEGAQEEGAQQPTPATAPTQGGVNSPVNVTTTVDANTSLAKIENAIRMISDQRANLGAFQNRLESIKNSTEYAIENLKASYAQIKDATMTDEVVAATTNSILTQSAMAMIAQANQVPQYVLSLLR

SEQ ID NO: 20 - protein, 336 aa, Borrelia afzelii
MIINHNTSAINASRNNAINAANLSKTQEKLSSGYRINRASDDAAGMGVSGKINAQIRGLSQASRNTSKAINFIQTTEGNLNEVEKVLVRMKELAVQSGNGTYSDSDRGSIQIEIEQLTDEINRIADQAQYNQMHMLSNKSASQNVKTAEELGMQPAKINTPASLSGSQASWTLRVHVGANQDEAIAVNIYSANVANLFAGEGAQAAQAAPVQEGAQEEGAQQPTPATAPTQGGVNSPVNVTTTVDANTSLAKIENAIRMISDQRANLGAFQNRLESIKNSTEYAIENLKASYAQIKDATMTDEVVAATTNSILTQSAMAMIAQANQVPQYVLSLLR

SEQ ID NO: 21 - protein, 339 aa, Borrelia burgdorferi
MNKILLLILLESIVFLSCSGKGSLGSEIPKVSLIIDGTFDDKSFNESALNGVKKVKEEFKIELVLKESSSNSYLSDLEGLKDAGSDLIWLIGYRFSDVAKVAALQNPDMKYAIIDPIYSNDPIPANLVGMTFRAQEGAFLTGYIAARLSKTGKIGFLGGIEGEIVDAFRYGYEAGAKYANKDIKIFTQYIGSFADLEAGRSVATRMYSDEIDIIHHAAGLGGIGAIEVAKELGSGHYIIGVDEDQAYLAPDNVITSTTKDVGRALNIFTSNHLKTNTFEGGKLINYGLKEGVVGFVRNPKMISFELEKEIDNLSSKIINKEIIVPSNKESYEKFLKEFI

SEQ ID NO: 22 - protein, 361 aa, Borrelia garinii
MHLIYLNQSYKLLRYNFIWKGEQFMNKSLLLILFECIIFLSCGGKGSLENEIPKVSLIIDGTFDDKSFNESALNGIKKVKEEFKIEPVLKESSINSYLSDLEGLKDTGSNLIWLIGYKFSDVAKAVSLQNPEIKYAIIDPVYSDEPIPANLVGMTFRSQEGAFLTGYIAAKVSKTGKIGFLGGIEGEIVDSFRYGYEAGAKYANKDIKISAYYIGSFADVEAGRSVATKMYSDGIDIIHHAAGLGGIGAIEVAKELGSGHYIIGVDEDQSYLAPNNIITSATKDVGRSLNIFTSNYLKTNTFEGGRLINYGLKEGVVGFVKNPKMIPFELEKEIDNLSSKIINKEIIVPYNKESYEKFLKE

SEQ ID NO: 23 - protein, 339 aa, Borrelia afzelii
MNKLLLLILFEGVIFLSCSGKSGLESGIPKVSLVIDGTFDDKSFNESALNGVKKLKEEFEIELVLKESSTNSYLSDLEGLKDAGSNLIWLIGYKFSDVAKAVSLQNSEMKYAIIDPVYSNEPIPSNLVGMTFRAQEGAFLTGYIAAKVSKTGKIGFLGGIEGDIVDAFRYGYEAGAKYANKDIKIFSQYIGSFSDLEAGRSVATKMYSDGIDIIHHAASLAGIGAIEVAKELGSGHYIIGVDEDQSYLAPNNVITSTTKDVGRSLNLFTSNYLKTNTFEGGKLINYGLKEGVVGFVRNPKMIPFEVEKEIDSLSSKIINKEVIVPYNKESYEKFLKEFI

(19) United States
(12) Patent Application Publication
(10) Pub. No.: US 2014/0274925 A1
(43) Pub. Date: Sep. 18, 2014

(54) Compositions and Methods for Diagnosing, Treating and Monitoring Lyme Disease

(71) Applicant: PHARMASAN LABS, INC., OSCEOLA, WI (US)

(72) Inventors: Chenggang Jin, Osceola, WI (US); Gottfried H. Kellermann, Osceola, WI (US)

(21) Appl. No.: 14/209,621

(22) Filed: Mar. 13, 2014

Related U.S. Application Data
(60) Provisional application No. 61/779,064, filed on Mar. 13, 2013.

(51) Int. Cl. G01N 33/569

(52) U.S. Cl. CPC G01N 33/56911
USPC 514/29; 435/7.24; 530/350; 514/152; 514/197; 514/202; 514/206; 514/207; 514/192

(57) ABSTRACT

Disclosed herein are compositions and methods for diagnosing, treating, and monitoring Lyme disease.

### CLAIM OF PRIORITY

This application claims priority to U.S. Patent Application Ser. No. 61/779,064, filed on Mar. 13, 2013, the entire contents of which are hereby incorporated by reference.

### TECHNICAL FIELD

This document provides methods and materials related to compositions and methods for diagnosing, treating, and monitoring treatment of Lyme disease.

### BACKGROUND

Lyme disease, or Lyme borreliosis, is the most prevalent tick-borne disease of humans in the United States. The Centers for Disease Control and Prevention (CDC) reported nearly 32,500 new cases in 2011, though it is estimated that the actual number is 10-fold higher, making Lyme disease an epidemic larger than AIDS, West Nile Virus, and Avian Flu combined.

Lyme disease is transmitted by the bite of blacklegged ticks. Infection is caused by a spirochete bacterium of the Borrelia genus, e.g., Borrelia burgdorferi, resulting in an illness affecting various organs of the body. The clinical implications of Lyme disease can be seen in dermatologic, neurologic and rheumatologic manifestations. While there is significant variability in the presentation of Lyme disease, typical symptoms include fever, headache, fatigue, swollen lymph nodes, muscle and joint aches, and sometimes a characteristic bull-eye shaped skin rash called “erythema migrans.”

Sometimes Lyme disease can be cured with antibiotic treatment alone, especially when the treatment begun early in the course of illness. Unfortunately only a fraction of Lyme patients are being treated due to equivocal clinical manifestations, inaccurate tests, and underreporting. The result of undiagnosed and untreated patients can lead to the development of a chronic Lyme infection or late stage Lyme diseases such as chronic Lyme arthritis or chronic Lyme neuroborreliosis, which can have devastating consequences in certain cases.

### SUMMARY

Provided herein are methods and materials for diagnosing, treating, and monitoring Lyme disease. The present invention is based in part on the development of a novel highly sensitive Lyme disease-specific enzyme-linked immunosorbent spot assay (Lyme ELISpot) that is capable of detecting T lymphocytes pre-sensitized to a Borrelia antigen at single cell resolution. High sensitivity of the Lyme ELISpot assay is ensured by a combination of serum-free medium, purified recombinant Lyme antigens, and/or co-stimulation by Interleukin-7 (IL-7). Provided herein are methods of using the Lyme ELISpot assays in the diagnosis of Borrelia infection and Lyme disease; and methods of monitoring the progress of treatment of Lyme disease. Compositions and kits comprising one or more Borrelia polypeptides, or antigenic fragments thereof, selected from the group consisting of but not limited to NapA, VlsE, DbpA, DbpB, OspC, OspA, OspB, P100, P41, P66, BmpA, BmpB, BmpC, Bgp, and Fbp, are also provided.

In one aspect, a method for diagnosing Lyme disease in a subject is provided. Peripheral blood mononuclear cells (PBMC) of the subject are provided and incubated in a serum-free medium with one or more Lyme antigens. The serum-free medium can also contain interleukin-7. The one or more Lyme antigens are polypeptides or proteins derived from or exhibiting sequence similarity to polypeptides or proteins derived from one or more pathogenic species of Borrelia: e.g., Borrelia burgdorferi sensu stricto, Borrelia afzelii, Borrelia garinii, Borrelia valaisiana, Borrelia bissettii, Borrelia Lusitaniae, and Borrelia spielmanii. The Lyme antigens can be native, recombinant, or synthetic. One or more cytokines secreted by the peripheral blood mononuclear cells can be measured by performing a bioassay, an immunoassay, a flow cytometry, or a radioimmunoassay (RIA).

Preferably, an enzyme-linked immunosorbent spot (ELISpot) assay is performed to measure the level of cytokines secreted by PBMC in response to Lyme antigen stimulation, and is designated as Lyme ELISpot assay. The cytokines measured by Lyme ELISpot can include IL-2, IL-3, IL-4, IL-5, IL-6, IL-9, IL-10, IL-13, IL-17A/F, IL-21, IL-22, IL-25, IL-31, TNF-α, TNF-β, IFN-γ, and GM-CSF. An increase in one or more cytokine level relative to a healthy control indicates that the subject may have Lyme disease.

Also provided herein are methods of treating Lyme disease in a subject. Such methods include (1) providing peripheral blood mononuclear cells (PBMCs) of the subject; (2) incubating the PBMCs in a serum-free medium with one or more Lyme antigens; (3) measuring the level of one or more cytokines secreted by the PBMCs; and (4) if the level of the one or more cytokines is above a control level, administering to the subject a treatment comprising one or more antibiotics. The control level can be the level of one or more cytokines secreted by the PBMCs in a healthy subject. The one or more antibiotics can be selected from doxycycline, amoxicillin, cefuroxime axetil, ceftriaxone, cefotaxime, penicillin, and azithromycin. The one or more antibiotics can be administered either orally or intravenously.

The Lyme antigens used to stimulate peripheral blood mononuclear cells can be a selection of one or more of the following polypeptides or antigenic fragments thereof: Variable major protein-like gene E (VlsE), Neutrophil activating protein (NapA), Decorin-binding protein A (DbpA), Decorin-binding protein B (DbpB), Outer surface protein C (OspC), Outer surface protein A (OspA), Outer surface protein B (OspB), P100, P41, P66, Borrelia membrane protein A (BmpA), Borrelia membrane protein B (BmpB), Borrelia membrane protein C (BmpC), Borrelia glycosaminoglycan-binding protein (Bgp), and Fibronectin-binding protein (Fbp).

In some embodiments, the Lyme antigen selection is a mixture of a NapA polypeptide or an antigenic fragment thereof, with one or more polypeptides or antigenic fragments thereof selected from VlsE, DbpA, DbpB, OspC, OspA, OspB, P100, P41, p66, BmpA, BmpB, BmpC, Bgp, and Fbp. In some embodiments, the Lyme antigen selection is a mixture of a NapA polypeptide or an antigenic fragment thereof, a DbpA polypeptide or an antigenic fragment thereof, an OspC polypeptide or an antigenic fragment thereof, a P100 polypeptide or an antigenic fragment thereof, and a VlsE polypeptide or an antigenic fragment thereof. In some embodiments, the Lyme antigen selection is a mixture of a NapA polypeptide or an antigenic fragment thereof, an OspC polypeptide or an antigenic fragment thereof, and a VlsE polypeptide or an antigenic fragment thereof.

In some embodiments, the Lyme antigen selection is a mixture of an OspA polypeptide or an antigenic fragment thereof, with one or more polypeptides or antigenic fragments thereof selected from NapA, VlsE, DbpA, DbpB, OspC, OspB, P100, P41, p66, BmpA, BmpB, BmpC, Bgp, and Fbp. In some embodiments, the Lyme antigen selection is a mixture of an OspA polypeptide or an antigenic fragment thereof, a DbpA polypeptide or an antigenic fragment thereof, an OspC polypeptide or an antigenic fragment thereof, a P100 polypeptide or an antigenic fragment thereof, and a VlsE polypeptide or an antigenic fragment thereof. In some embodiments, the Lyme antigen selection is a mixture of an OspA polypeptide or an antigenic fragment thereof, an OspC polypeptide or an antigenic fragment thereof, and a VlsE polypeptide or an antigenic fragment thereof.

In some embodiments, the Lyme antigen selection is a mixture of a NapA polypeptide or an antigenic fragment thereof, a DbpA polypeptide or an antigenic fragment thereof, an OspC polypeptide or an antigenic fragment thereof, a P100 polypeptide or an antigenic fragment thereof, a VlsE polypeptide or an antigenic fragment thereof, and an OspA polypeptide or an antigenic fragment thereof. In some embodiments, the Lyme antigen selection is a mixture of a DbpA polypeptide or an antigenic fragment thereof, an OspC polypeptide or an antigenic fragment thereof, a P100 polypeptide or an antigenic fragment thereof, and a VlsE polypeptide or an antigenic fragment thereof. In some embodiments, the Lyme antigen selection is a mixture of an OspC polypeptide or an antigenic fragment thereof and a VlsE polypeptide or an antigenic fragment thereof.

In some embodiments, an OspC polypeptide comprises an amino acid sequence having at least 80% sequence identity to any one of SEQ ID NOs:1-4, or to an antigenic fragment thereof. For example, an OspC polypeptide can have an amino acid sequence of any one of SEQ ID NOs:1-4.

In some embodiments, a P100 polypeptide comprises an amino acid sequence having at least 80% sequence identity to SEQ ID NO:5, or to an antigenic fragment thereof. For example, a P100 polypeptide can have an amino acid sequence of SEQ ID NO:5.

In some embodiments, a VlsE polypeptide comprises an amino acid sequence having at least 80% sequence identity to any one of SEQ ID NOs:6-7, or to an antigenic fragment thereof For example, a VlsE polypeptide can have an amino acid sequence of any one of SEQ ID NOs:6-7.

In some embodiments, a DbpA polypeptide comprises an amino acid sequence having at least 80% sequence identity to any one of SEQ ID NOs:8-10, or to an antigenic fragment thereof. For example, a DbpA polypeptide can have an amino acid sequence of any one of SEQ ID NOs:8-10.

In some embodiments, a DbpB polypeptide comprises an amino acid sequence having at least 80% sequence identity to any one of SEQ ID NOs:11-13, or to an antigenic fragment thereof. For example, a DbpB polypeptide can have an amino acid sequence of any one of SEQ ID NOs:11-13.

In some embodiments, a NapA polypeptide comprises an amino acid sequence having at least 80% sequence identity to any one of SEQ ID NOs:14-15, or to an antigenic fragment thereof. For example, a NapA polypeptide can have an amino acid sequence of any one of SEQ ID NOs:14-15.

In some embodiments, an OspA polypeptide comprises an amino acid sequence having at least 80% sequence identity to any one of SEQ ID NOs:16-18, or to an antigenic fragment thereof. For example, an OspA polypeptide can have an amino acid sequence of any one of SEQ ID NOs:16-18.

In some embodiments, a P41 polypeptide comprises an amino acid sequence having at least 80% sequence identity to any one of SEQ ID NOs:19-20, or to an antigenic fragment thereof. For example, a P41 polypeptide has an amino acid sequence of any one of SEQ ID NOs:19-20.

In some embodiments, a BmpA polypeptide comprises an amino acid sequence having at least 80% sequence identity to any one of SEQ ID NOs:21-23, or to an antigenic fragment thereof. For example, a BmpA polypeptide has an amino acid sequence of any one of SEQ ID NOs:21-23.

In some embodiments, the method for diagnosing Lyme disease in a subject can also include observing a Lyme disease related symptom in a subject, e.g., a tick bite, erythema migrans, skin lesion, pain, fever, headache, and/or swelling, or measuring a Lyme antigen specific antibody in a blood sample of the subject using Western blot or enzyme-linked immunosorbent assay (ELISA). In some embodiments, Lyme ELISpot assays can be used in combination with other assays to differentiate active ongoing Borrelia infection from a previous or latent Borrelia infection.

In another aspect, a method for monitoring a treatment of Lyme disease in a subject is provided. In some embodiments, a first sample of peripheral blood mononuclear cells (PBMC) can be obtained from a subject before receiving a treatment, and a first level of one or more cytokines secreted by the PBMC can be measured by Lyme ELISpot assay. A second sample of PBMC can be obtained from the subject during or after a treatment of Lyme disease, and a second level of one or more cytokines secreted by the second sample of PBMC can be measured by Lyme ELISpot assay. The first level and the second level can then be compared to assess the effectiveness of the treatment. If the second level is lower than the first level, the treatment may be effective for the subject. Conversely, if the second level is higher than or similar to the first level, the treatment may be ineffective for the subject. In some embodiments, the method for monitoring a treatment of Lyme disease in a subject can also include observing Lyme disease related symptom in a subject, e.g., a tick bite, erythema migrans, skin lesion, pain, fever, headache, and/or swelling, or measuring a Lyme antigen specific antibody in a blood sample of the subject using Western blot or ELISA before and after treatment.

In another aspect, a composition is provided. The composition include one or more Lyme antigen polypeptides or antigenic fragments thereof selected from the group consisting of a VlsE polypeptide or an antigenic fragment thereof, a NapA polypeptide or an antigenic fragment thereof, a DbpA polypeptide or an antigenic fragment thereof, a DbpB polypeptide or an antigenic fragment thereof, an OspC polypeptide or an antigenic fragment thereof, an OspA polypeptide or an antigenic fragment thereof, an OspB polypeptide or an antigenic fragment thereof, a P100 polypeptide or an antigenic fragment thereof, a P41 polypeptide or an antigenic fragment thereof, a P66 polypeptide or an antigenic fragment thereof, a BmpA polypeptide or an antigenic fragment thereof, a BmpB polypeptide or an antigenic fragment thereof, a BmpC polypeptide or an antigenic fragment thereof, a Bgp polypeptide or an antigenic fragment thereof, and a Fbp polypeptide or an antigenic fragment thereof. The one or more Lyme antigen polypeptides can be native, recombinant or synthetic polypeptides.

In some embodiments, the composition is a mixture of a NapA polypeptide or an antigenic fragment thereof, with one or more polypeptides or antigenic fragments thereof selected from VlsE, DbpA, DbpB, OspC, OspA, OspB, P100, P41, p66, BmpA, BmpB, BmpC, Bgp, and Fbp. In some embodiments, the composition is a mixture of a NapA polypeptide or an antigenic fragment thereof, a DbpA polypeptide or an antigenic fragment thereof, an OspC polypeptide or an antigenic fragment thereof, a P100 polypeptide or an antigenic fragment thereof, and a Vlse polypeptide or an antigenic fragment thereof. In some embodiments, the composition is a mixture of a NapA polypeptide or an antigenic fragment thereof, an OspC polypeptide or an antigenic fragment thereof, and a VlsE polypeptide or an antigenic fragment thereof.

In some embodiments, the composition is a mixture of an OspA polypeptide or an antigenic fragment thereof, with one or more polypeptides or antigenic fragments thereof selected from NapA, VlsE, DbpA, DbpB, OspC, OspB, P100, P41, p66, BmpA, BmpB, BmpC, Bgp, and Fbp. In some embodiments, the composition is a mixture of an OspA polypeptide or an antigenic fragment thereof, a DbpA polypeptide or an antigenic fragment thereof, an OspC polypeptide or an antigenic fragment thereof, a P100 polypeptide or an antigenic fragment thereof, and a Vlse polypeptide or an antigenic fragment thereof. In some embodiments, the composition is a mixture of an OspA polypeptide or an antigenic fragment thereof, an OspC polypeptide or an antigenic fragment thereof, and a VlsE polypeptide or an antigenic fragment thereof.

In some embodiments, the composition is a mixture of a NapA polypeptide or an antigenic fragment thereof, a DbpA polypeptide or an antigenic fragment thereof, an OspC polypeptide or an antigenic fragment thereof, a P100 polypeptide or an antigenic fragment thereof, a Vlse polypeptide or an antigenic fragment thereof, and an OspA polypeptide or an antigenic fragment thereof. In some embodiments, the composition is a mixture of a DbpA polypeptide or an antigenic fragment thereof, an OspC polypeptide or an antigenic fragment thereof, a P100 polypeptide or an antigenic fragment thereof, and a VlsE polypeptide or an antigenic fragment thereof. In some embodiments, the composition is a mixture of an OspC polypeptide or an antigenic fragment thereof and a VlsE polypeptide or an antigenic fragment thereof.

In some embodiments, an OspC polypeptide in the composition comprises an amino acid sequence having at least 80% sequence identity to any one of SEQ ID NOs:1-4, or to an antigenic fragment thereof. For example, an OspC polypeptide can have an amino acid sequence of any one of SEQ ID NOs:1-4.

In some embodiments, a P100 polypeptide in the composition comprises an amino acid sequence having at least 80% sequence identity to SEQ ID NO:5, or to an antigenic fragment thereof. For example, a P100 polypeptide can have an amino acid sequence of SEQ ID NO:5.

In some embodiments, a VlsE polypeptide in the composition comprises an amino acid sequence having at least 80% sequence identity to any one of SEQ ID NOs:6-7, or to an antigenic fragment thereof. For example, a VlsE polypeptide can have an amino acid sequence of any one of SEQ ID NOs:6-7.

In some embodiments, a DbpA polypeptide in the composition comprises an amino acid sequence having at least 80% sequence identity to any one of SEQ ID NOs:8-10, or to an antigenic fragment thereof. For example, a DbpA polypeptide can have an amino acid sequence of any one of SEQ ID NOs:8-10.

In some embodiments, a DbpB polypeptide in the composition comprises an amino acid sequence having at least 80% sequence identity to any one of SEQ ID NOs:11-13, or to an antigenic fragment thereof. For example, a DbpB polypeptide can have an amino acid sequence of any one of SEQ ID NOs:11-13.

In some embodiments, a NapA polypeptide in the composition comprises an amino acid sequence having at least 80% sequence identity to any one of SEQ ID NOs:14-15, or to an antigenic fragment thereof. For example, a NapA polypeptide can have an amino acid sequence of any one of SEQ ID NOs:14-15.

In some embodiments, an OspA polypeptide in the composition comprises an amino acid sequence having at least 80% sequence identity to any one of SEQ ID NOs:16-18, or to an antigenic fragment thereof. For example, an OspA polypeptide can have an amino acid sequence of any one of SEQ ID NOs:16-18.

In some embodiments, a P41 polypeptide in the composition comprises an amino acid sequence having at least 80% sequence identity to any one of SEQ ID NOs:19-20, or to an antigenic fragment thereof. For example, a P41 polypeptide has an amino acid sequence of any one of SEQ ID NOs:19-20.

In some embodiments, a BmpA polypeptide in the composition comprises an amino acid sequence having at least 80% sequence identity to any one of SEQ ID NOs:21-23, or to an antigenic fragment thereof. For example, a BmpA polypeptide has an amino acid sequence of any one of SEQ ID NOs:21-23.

In yet another aspect, a kit comprising the composition is provided. The kit can also include instructions for use and other reagents such as serum-free medium; IL-7; a positive control, e.g., phytohaemagglutinin (PHA); microtiter plates coated with one or more capture antibodies specific to a cytokine; a detection antibody to the cytokine; a chromogenic, fluorogenic, or electrochemiluminescent substrate; buffers and antimicrobial agents. The capture and detection antibodies can be monoclonal or polyclonal antibodies that bind to different epitopes on the cytokine. The detection antibody can be any detectably labeled antibody, for example, an antibody tagged with a fluorescent dye, an enzyme-conjugated antibody, or an antibody conjugated with one member of a specific binding pair, e.g., an antibody conjugated with biotin or streptavidin. For example, when a biotinylated detection antibody is included in the kit, the kit also includes enzyme-conjugated streptavidin.

The term “Lyme antigen” used herein refers to polypeptides or proteins derived from or exhibiting sequence similarity to polypeptides or proteins derived from one or more pathogenic species of Borrelia: e.g., Borrelia burgdorferi sensu stricto, Borrelia afzelii, Borrelia garinii, Borrelia valaisiana, Borrelia bissettii, Borrelia lusitaniae, and Borrelia spielmanii. The terms “polypeptide” and “protein” are used interchangeably herein and refer to any chain of amino acids, regardless of length or post-translational modification (e.g., glycosylation or phosphorylation).

The term “antigenic fragment” used herein refers to a portion of a polypeptide capable of binding to a major histocompatibility complex (MHC) and being presented to a T-cell receptor.

The term “mixture” or “antigen mixture” used herein refers to a composition comprising at least two Lyme antigen polypeptides or proteins.

As used herein, the term “percent sequence identity” refers to the degree of identity between any given query sequence and a subject sequence. Percentage of “sequence identity” is determined by comparing two optimally aligned sequences over a comparison window, where the fragment of the amino acid sequence in the comparison window may comprise additions or deletions (e.g., gaps or overhangs) as compared to the reference sequence (which does not comprise additions or deletions) for optimal alignment of the two sequences. The percentage is calculated by determining the number of positions at which the identical amino acid residue occurs in both sequences to yield the number of matched positions, dividing the number of matched positions by the total number of positions in the window of comparison and multiplying the result by 100 to yield the percentage of sequence identity. The output is the percent identity of the subject sequence with respect to the query sequence. It is noted that a query nucleotide or amino acid sequence that aligns with a subject sequence can result in many different lengths, with each length having its own percent identity.

As used herein, a “subject” is an animal, e.g., a mammal, e.g., a human, monkey, dog, cat, horse, cow, pig, goat, rabbit, or mouse.

As used herein, the term “treat” or “treatment” is defined as the application or administration of a treatment regimen, e.g., a therapeutic agent or modality, to a subject. The treatment can be to cure, heal, alleviate, relieve, alter, remedy, ameliorate, palliate, improve or affect Lyme disease or symptoms associated with Lyme disease.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 is a representative Lyme ELISpot report of a Lyme positive patient showing Lyme antigen NapA titration. Tests were run in triplicate for each condition. Serum-free medium alone serves as a negative control, and phytohaemagglutinin (PHA) serves as a positive control.

FIG. 2 is a representative Lyme ELISpot report of a Lyme positive patient showing Lyme antigen OspA titration. Tests were run in triplicate for each condition. Serum-free medium alone serves as a negative control, and PHA serves as a positive control.

FIG. 3 is a representative Lyme ELISpot report of a Lyme antigen negative donor. Both the Lyme ELISpot assay with IL-7 and the Lyme ELISpot assay without IL-7 reported negative results. Serum-free medium alone serves as a negative control, and PHA (phytohaemagglutinin) serves as a positive control. Antigen mix 1 is a mixture of recombinant Lyme antigens DbpA, OspC, p100 and VlsE, and Antigen mix 2 is a mixture of recombinant Lyme antigens OspC and VlsE.

FIG. 4 is a representative Lyme ELISpot report showing the positive results in a Lyme antigen positive donor. While both the Lyme ELISpot assay with IL-7 and the Lyme ELISpot assay without IL-7 reported positive results, the addition of IL-7 dramatically amplified the signals of Lyme antigen-positive cells.

FIG. 5 is a representative Lyme ELISpot report of a Lyme antigen positive donor showing the different results reported by the Lyme ELISpot assay with IL-7 and by the Lyme ELISpot assay without IL-7. While the Lyme ELISpot assay without IL-7 reported negative results, the Lyme ELISpot assay with IL-7 reported positive results, indicating that the addition of IL-7 improved the detection of Lyme antigen-specific cells.

FIGS. 6A and 6B are a bar graph (6A) and a dot plot (6B) showing that the Lyme ELISpot with IL-7 results are significantly (P=0.017) different from the Lyme ELISpot results without IL-7.

FIG. 7A is a dot plot showing the frequency of Lyme antigen-induced IFN-γ spot in PBMC of Borrelia positive patients formed in either Lyme ELISpot assay using Lyme Antigen mix 1 and IL-7 (the iSpot Lyme assays), or Lyme ELISpot assay using Lyme Antigen mix 2 but not IL-7. Data points obtained from the same donor are connected by a line. Each data point represents the mean spot forming unit (SFU) of triplicate Lyme antigen-stimulated wells minus the mean SFU of the corresponding medium control wells. A non-parametric Mann-Whitney U test was used to compare the matched results with a p-value of <0.05 considered statistically significant.

FIG. 7B is a dot plot showing the size distribution of IFN-γ ELISpots formed in either the iSpot Lyme assay (closed circles), or the Lyme ELISpot assay using Antigen mix 2 but not IL-7 (open circles).

FIG. 8A is a dot plot showing the iSpot Lyme assay results performed on 80 healthy controls (HC), 25 clinically diagnosed Lyme disease patients (LD), and 23 non-Lyme patients (NLP, patients with other clinical conditions but not Lyme disease). Each symbol represents the mean SFU obtained from triplicate test wells stimulated with Lyme antigen after subtraction of the mean SFU in triplicate control wells. Non-parametric Mann-Whitney U test was used to compare the results between LD and HC, and between LD and NLP. A p-value <0.05 was considered statistically significant. The dotted line represents the cutoff value for positivity at 25 SFU.

FIGS. 8B and 8C are line graphs showing the Receiver Operating Characteristics (ROC) curve of the iSpot Lyme assay (8B) or the Lyme ELISpot assay using Antigen mix 2 but not IL-7 (8C). The ROC analysis was used to determine the sensitivity, specificity, positive predictive value (PPV), negative predictive value (NPV), area under the curve value (AUC) and cutoff value for both Lyme ELISpot assays.

FIG. 9A is a bar graph showing the intra-assay precision of the iSpot Lyme assays. Five PBMC samples with different Lyme antigen-triggered SFU response levels were each tested in triplicate wells. Bars with the specified shades show the reactivity for the three individual wells and the average of these three wells. The coefficient of variation for the replicate wells was labeled above the corresponding bars.

FIG. 9B is a bar graph showing the inter-assay precision of the iSpot Lyme assay. Cryopreserved PBMC aliquots of each of the three specified Lyme patients were tested for Borrelia reactivity on five consecutive days. The coefficient of variation for inter assay variation was was labeled above the corresponding bars.

FIGS. 9C and 9D are line graphs showing the relationship between the number of PBMC plated in each well and the IFN-γ SFU value in a Borrelia positive subject (9C) or a healthy control (9D). Open symbols in 9D represent the mean of triplicate test wells treated with Lyme antigens, while the closed symbols in 9D represent the mean of the corresponding control wells treated with medium. The Standard Deviation (SD) for the triplicate is smaller than the symbol when not visible.

FIG. 9E is a line graph showing the dose response curve of the Lyme antigen mix for the iSpot Lyme assay and the Lyme ELISpot assay using Lyme Antigen mix 2 but not IL-7 (labeled as ELISPOT).

FIG. 9F is a line graph showing the correlation of the frequency of IFN-γ secreting Borrelia-specific T cells as measured by the iSpot Lyme assay and the concentrations of IFN-γ in the culture supernatant as measured by the Bio-Plex suspension array. The nonparametric Spearman's test was used to determine the correlation. The results showed a p-value <0.0001.

FIG. 10A is a bar graph showing the sensitivity difference among Western blot, the Lyme ELISpot assay using antigen mix 2 but not IL-7, and the iSpot Lyme assay. The sensitivity was determined by a clinical study of 23 diagnosed Lyme patients, including both seropositive and seronegative patients.

FIG. 10B is a bar graph showing the sensitivity difference among Western blot, the iSpot Lyme assay, and the Lyme ELISpot assay using Lyme Antigen mix 2 but not IL-7 in 17 diagnosed seronegative Lyme patients.

FIG. 10C is a bar graph showing the cross-reactivity of the iSpot Lyme assay, the Lyme ELISpot assay using Lyme Antigen mix 2 but not IL-7, and the Western Blot assay in 23 subjects with other clinical conditions but not Lyme disease.

FIG. 11 contains amino acid sequences for Lyme antigens set forth as SEQ ID NOs:1-23.

### DETAILED DESCRIPTION

Diagnosis of Lyme disease is often based upon a physician's review of clinical symptoms and the patient's exposure risk in an area where the disease is endemic. Prompt diagnosis and treatment of Lyme disease is the key to avoiding chronic Lyme disease and its deleterious effects. Early detection of Lyme disease can be difficult because the characteristic rash may not be present and the flu-like symptoms can be caused by many other factors which can confuse diagnosis.

For laboratory tests, the Centers for Disease Control (CDC) recommend a two-tiered approach consisting of an enzyme linked immunosorbent assay (ELISA) and a Western Blot (WB), both of which are serological assays that detect antibodies specific to an antigen of the Borrelia bacteria. The sensitivity of the two-tiered tests, however, is only about 30% in early Lyme disease and 50% in late Lyme disease. Moreover, the two-tiered tests report false-negative results for seronegative Lyme patients, consisting of about 30-50% of all Lyme patients.

Disclosed herein are methods and materials for diagnosing, treating, and monitoring Lyme disease. The present invention is based in part on the development of a novel highly sensitive Lyme disease-specific enzyme-linked immunosorbent spot assay (Lyme ELISpot) that is capable of detecting T lymphocytes pre-sensitized to a Borrelia antigen at single cell resolution. High sensitivity of the Lyme ELISpot assay is ensured by a c of serum-free medium, purified recombinant Lyme antigens, and/or co-stimulation by Interleukin-7 (IL-7). Provided herein are methods of using the Lyme ELISpot assays in the diagnosis of Borrelia infection and Lyme disease; methods of monitoring the progress of treatment of Lyme disease. Compositions and kits comprising one or more Borrelia polypeptides, or antigenic fragments thereof, selected from the group consisting of but not limited to NapA, VlsE, DbpA, DbpB, OspC, OspA, OspB, P100, P41, P66, BmpA, BmpB, BmpC, Bgp, and Fbp, are also provided.

### Lyme Antigens

Lyme antigens are polypeptides or proteins having one or more immunoreactive epitopes, which are derived from or exhibiting sequence similarity to polypeptides or proteins derived from one or more pathogenic species of Borrelia: e.g., Borrelia burgdorferi sensu stricto, Borrelia afzelii, Borrelia garinii, Borrelia valaisiana, Borrelia bissettii, Borrelia lusitaniae, and Borrelia spielmanii (Chu et al., Journal of Medical Microbiology 57: 980-985, 2008). The terms “polypeptide” and “protein” are used interchangeably herein and refer to any peptide-linked chain of amino acids, regardless of length or post-translational modification. A polypeptide for use in the materials and methods described herein can be an antigenic fragment of any of the polypeptides described herein, provided the antigenic fragment includes at least one epitope of the reference polypeptide.

By way of example and without limitation, a polypeptide can be a native, recombinant, or chemically synthesized polypeptide or antigenic fragment thereof. In some embodiments, a polypeptide can be a substantially purified polypeptide obtained from a whole organism lysate. A substantially purified polypeptide or protein is substantially free of cellular material or other contaminating proteins from the cell or tissue source from which the protein is derived, or substantially free from chemical precursors or other chemicals when chemically synthesized. Any appropriate method for obtaining substantially pure polypeptides can be used.

In some embodiments, a polypeptide can be obtained by expression of a recombinant nucleic acid encoding the polypeptide or by chemical synthesis (e.g., by solid-phase synthesis or other methods well known in the art, including synthesis with an ABI peptide synthesizer; Applied Biosystems, Foster City, Calif.). Expression vectors that encode the polypeptide of interest can be used to produce a polypeptide. For example, standard recombinant technology using expression vectors encoding a polypeptide can be used. Expression systems that can be used for small or large-scale production of the polypeptides provided herein include, without limitation, microorganisms such as bacteria transformed with recombinant bacteriophage DNA, plasmid DNA, or cosmid DNA expression vectors containing the nucleic acid molecules of the polypeptide of interest. The resulting polypeptides can be purified according to any appropriate protein purification method. In some embodiments, substantially pure polypeptides or antigenic fragments thereof can be purchased from a commercial supplier (e.g., Diarect, Freiburg, Germany).

Antigens appropriate for the compositions and methods provided herein can be recombinant or synthetic polypeptides exhibiting a percent sequence identity to the native polypeptides derived from one or more species of Borrelia. As used herein, the term “percent sequence identity” refers to the degree of identity between any given query sequence and a subject sequence. Percentage of “sequence identity” is determined by comparing two optimally aligned sequences over a comparison window, where the fragment of the amino acid sequence in the comparison window may comprise additions or deletions (e.g., gaps or overhangs) as compared to the reference sequence (which does not comprise additions or deletions) for optimal alignment of the two sequences. The percentage is calculated by determining the number of positions at which the identical amino acid residue occurs in both sequences to yield the number of matched positions, dividing the number of matched positions by the total number of positions in the window of comparison and multiplying the result by 100 to yield the percentage of sequence identity. The output is the percent identity of the subject sequence with respect to the query sequence. It is noted that a query nucleotide or amino acid sequence that aligns with a subject sequence can result in many different lengths, with each length having its own percent identity. Optimal alignment of sequences for comparison may be conducted by the local homology algorithm of Smith and Waterman (1981) Add. APL. Math. 2:482; by the homology alignment algorithm of Needleman and Wunsch (1970) J. Mol. Biol. 48:443; by the search for similarity method of Pearson and Lipman (1988) Proc. Natl. Acad. Sci. (USA) 85: 2444; by computerized implementations of algorithms such as GAP, BESTFIT, BLAST, PASTA, and TFASTA (Accelrys, Inc., 10188 Telesis Court, Suite 100 San Diego, Calif. 92121); or by inspection. Typically, the default values of 5.00 for gap weight and 0.30 for gap weight length are used.

The following Lyme antigens are provided to demonstrate the utility of the current testing platform. OspC is an outer surface protein expressed as the spirochete traverses to the mammalian host, whereas related outer surface polypeptides OspA and OspB are mainly expressed in the mid-gut of the tick. Amino acid sequences of OspC proteins from Borrelia burgdorferi, Borrelia valaisiana, Borrelia garinii, and Borrelia afzelii are set forth in SEQ ID NOs:1-4, respectively. A Lyme antigen for inclusion in a composition described herein can include an amino acid sequence having at least 80% (e.g., 80%, 85%, 90%, 95%, 100%) sequence identity to a Borrelia OspC polypeptide, or to an antigenic fragment thereof. In some embodiments, a Lyme antigen can include an amino acid sequence having at least 80% (e.g., 80%, 85%, 90%, 95%, 100%) identity to a polypeptide selected from SEQ ID NOs:1-4, or to an antigenic fragment thereof. In some embodiments, a Lyme antigen has an amino acid sequence of SEQ ID NO:1, 2, 3 or 4.

Amino acid sequences of OspA proteins from Borrelia burgdorferi, Borrelia valaisiana, Borrelia garinii, and Borrelia afzelii are set forth in SEQ ID NOs:16-18, respectively. A Lyme antigen for inclusion in a composition described herein can include an amino acid sequence having at least 80% (e.g., 80%, 85%, 90%, 95%, 100%) sequence identity to a Borrelia OspA polypeptide, or to an antigenic fragment thereof. In some embodiments, a Lyme antigen can include an amino acid sequence having at least 80% (e.g., 80%, 85%, 90%, 95%, 100%) identity to a polypeptide selected from SEQ ID NOs:16-18, or to an antigenic fragment thereof. In some embodiments, a Lyme antigen has an amino acid sequence of SEQ ID NO:16, 17, or 18.

P100 is a high molecular weight major antigen of the membranous vesicle on the surface of Borrelia burgdorferi and is expressed late in Borrelia infection. Antibodies against P100 are usually of the IgG type and generally only appear in the chronic stage of the infection. Amino acid sequences of P100 protein from Borrelia burgdorferi is set forth in SEQ ID NO:5. A Lyme antigen for inclusion in a composition described herein can include an amino acid sequence having at least 80% (e.g., 80%, 85%, 90%, 95%, 100%) sequence identity to a Borrelia P100 polypeptide, or to an antigenic fragment thereof. In some embodiments, a Lyme antigen can include an amino acid sequence having at least 80% (e.g., 80%, 85%, 90%, 95%, 100%) identity to polypeptide SEQ ID NO:5, or to an antigenic fragment thereof. In some embodiments, a Lyme antigen has an amino acid sequence of SEQ ID NO:5, or an antigenic fragment thereof.

Variable major protein-like E (VlsE) is an outer surface lipoprotein that undergoes antigenic variation during disseminated infection. The Borrelia bacterium is hidden from the immune system by antigenic variation of surface proteins expressed by VlsE genes. Thus, antibodies to VlsE can serve as a diagnostic marker of later stages of Borrelia infection. Amino acid sequences of VlsE proteins from Borrelia burgdorferi and Borrelia garinii are set forth in SEQ ID NOs:6-7, respectively. A Lyme antigen for inclusion in a composition described herein can include an amino acid sequence having at least 80% (e.g., 80%, 85%, 90%, 95%, 100%) sequence identity to a Borrelia VlsE polypeptide, or to an antigenic fragment thereof. In some embodiments, a Lyme antigen can include an amino acid sequence having at least 80% (e.g., 80%, 85%, 90%, 95%, 100%) identity to a polypeptide selected from SEQ ID NOs:6-7, or to an antigenic fragment thereof. In some embodiments, a Lyme antigen has an amino acid sequence of SEQ ID NO: 6 or 7.

P41, or flagellin, is expressed in early and late Borrelia infection. Amino acid sequences of P41/flagellin proteins from Borrelia afzelii and Borrelia burgdorferi are set forth in SEQ ID NOs: 19-20. A Lyme antigen for inclusion in a composition described herein can include an amino acid sequence having at least 80% (e.g., 80%, 85%, 90%, 95%, 100%) sequence identity to a Borrelia P41/flagellin polypeptide, or to an antigenic fragment thereof. In some embodiments, a Lyme antigen can include an amino acid sequence having at least 80% (e.g., 80%, 85%, 90%, 95%, 100%) identity to a polypeptide selected from SEQ ID NOs:19-20, or to an antigenic fragment thereof. In some embodiments, a Lyme antigen has an amino acid sequence of SEQ ID NO:19 or 20.

Additional antigens appropriate for the compositions provided herein are bacterial antigens that bind host proteins. For example, BmpA is a Borrelia membrane protein that enhances spirochete colonization and survival in host tissues. BmpA and its three paralogous proteins, BmpB, BmpC, and BmpD, bind mammalian laminin. Accordingly, polypeptides suitable for the compositions provided herein can have an amino acid sequence with at least 80% (e.g., 80%, 85%, 90%, 95%, 100%) sequence identity to a Borrelia BmpA, BmpB, BmpC, or BmpD protein. In some embodiments, a Lyme antigen for inclusion in a composition described herein can include an amino acid sequence having at least 80% (e.g., 80%, 85%, 90%, 95%, 100%) sequence identity to BmpA amino acid sequences set forth as SEQ ID NO:21 (Borrelia burgdorferi), SEQ ID NO:22 (Borrelia garinii), or SEQ ID NO:23 (Borrelia afzelii). In some embodiments, a Lyme antigen has an amino acid sequence of SEQ ID NO:21, 22, or 23.

Decorin-binding proteins A and B (DbpA and DbpB) bind decorin, a proteoglycan that associates with collagen. The decorin binding proteins promote binding of the spirochete to extracellular matrix proteins of host cells for maximum colonization of host tissues including skin and joints. Accordingly, polypeptides suitable for the compositions provided herein can have an amino acid sequence with at least 80% (e.g., 80%, 85%, 90%, 95%, 100%) sequence identity to a Borrelia DbpA. In some embodiments, a Lyme antigen for inclusion in a composition described herein can include an amino acid sequence having at least 80% (e.g., 80%, 85%, 90%, 95%, 100%) sequence identity to DbpA amino acid sequences set forth as SEQ ID NO:8 (Borrelia burgdorferi), SEQ ID NO:9 (Borrelia garinii), or SEQ ID NO:10 (Borrelia afzelii). In some embodiments, a Lyme antigen for inclusion in a composition described herein can include an amino acid sequence having at least 80% (e.g., 80%, 85%, 90%, 95%, 100%) sequence identity to DbpB amino acid sequences set forth as SEQ ID NO:11 (Borrelia burgdorferi), SEQ ID NO:12 (Borrelia garinii), or SEQ ID NO:13 (Borrelia afzelii). In some embodiments, a Lyme antigen has an amino acid sequence of SEQ ID NO:11, 12, or 14.

Neutrophil activating protein (NapA) is a member of the Dps-like protein family with specific immunomodulatory properties. In particular, NapA can induce the expression of IL-23 in neutrophils and monocytes, as well as the expression of IL-6, IL-1β, and transforming growth factor β (TGF-β) in monocytes, via Toll-like receptor 2 (TLR2). NapA is the main Borrelia product involved in the pathogenesis of Lyme arthritis through accumulating and orchestrating the recruitment of inflammatory cells into the joint cavity. Accordingly, polypeptides suitable for the compositions provided herein can have an amino acid sequence with at least 80% (e.g., 80%, 85%, 90%, 95%, 100%) sequence identity to a Borrelia NapA. In some embodiments, a Lyme antigen for inclusion in a composition described herein can include an amino acid sequence having at least 80% (e.g., 80%, 85%, 90%, 95%, 100%) sequence identity to NapA amino acid sequences set forth as SEQ ID NO:14 (Borrelia burgdorferi), or SEQ ID NO:15 (Borrelia afzelii). In some embodiments, a Lyme antigen has an amino acid sequence of SEQ ID NO:14 or 15.

Other host receptor binding proteins can include P66, a 66-kilodalton (kD) spirochetal polypeptide that binds platelet-specific integrin α2bβ3 and the vitronectin receptor αvβ3; Bgp, a 26-kD glycosaminoglycan-binding polypeptide that binds heparin sulfate and dermatin sulphate; and Fbp, a 47 kD fibronectin-binding polypeptide. Accordingly, polypeptides exhibiting at least 80% (e.g., 80%, 85%, 90%, 95%, 100%) sequence identity to Borrelia P66, Bgp, or Fbp polypeptides are also suitable for inclusion.

### Compositions

Compositions provided herein typically include one or more (e.g., 1, 2, 3, 4, 5, 6, or more) Lyme antigens selected from the group consisting of but not limited to a VlsE polypeptide or an antigenic fragment thereof, a NapA polypeptide or an antigenic fragment thereof, a DbpA polypeptide or an antigenic fragment thereof, a DbpB polypeptide or an antigenic fragment thereof, an OspC polypeptide or an antigenic fragment thereof, an OspA polypeptide or an antigenic fragment thereof, an OspB polypeptide or an antigenic fragment thereof, a P100 polypeptide or an antigenic fragment thereof, a P41 polypeptide or an antigenic fragment thereof, a P66 polypeptide or an antigenic fragment thereof, a BmpA polypeptide or an antigenic fragment thereof, a BmpB polypeptide or an antigenic fragment thereof, a BmpC polypeptide or an antigenic fragment thereof, a Bgp polypeptide or an antigenic fragment thereof, and a Fbp polypeptide or an antigenic fragment thereof. The polypeptides or antigenic fragment thereof can be native, recombinant, or chemically synthesized.

In some embodiments, the compositions provided herein can include a mixture of an OspC polypeptide or an antigenic fragment thereof and a VlsE polypeptide or an antigenic fragment thereof. In some embodiments, the compositions provided herein can include a mixture of a DbpA polypeptide or an antigenic fragment thereof, an OspC polypeptide or an antigenic fragment thereof, a P100 polypeptide or an antigenic fragment thereof, and a VlsE polypeptide or an antigenic fragment thereof.

In some embodiments, the compositions provided herein can include a mixture of a NapA polypeptide or an antigenic fragment thereof, with one or more polypeptides or antigenic fragments thereof selected from VlsE, DbpA, DbpB, OspC, OspA, OspB, P100, P41, P66, BmpA, BmpB, BmpC, Bgp, and Fbp. In some embodiments, the compositions provided herein can include a mixture of a NapA polypeptide or an antigenic fragment thereof, an OspC polypeptide or an antigenic fragment thereof and a VlsE polypeptide or an antigenic fragment thereof. In some embodiments, the compositions provided herein can include a mixture of a NapA polypeptide or an antigenic fragment thereof, a DbpA polypeptide or an antigenic fragment thereof, an OspC polypeptide or an antigenic fragment thereof, a P100 polypeptide or an antigenic fragment thereof, and a VlsE polypeptide or an antigenic fragment thereof.

In some embodiments, the compositions provided herein can include a mixture of an OspA polypeptide or an antigenic fragment thereof, with one or more polypeptides or antigenic fragments thereof selected from NapA, VlsE, DbpA, DbpB, OspC, OspB, P100, P41, P66, BmpA, BmpB, BmpC, Bgp, and Fbp. In some embodiments, the compositions provided herein can include a mixture of an OspA polypeptide or an antigenic fragment thereof, an OspC polypeptide or an antigenic fragment thereof and a VlsE polypeptide or an antigenic fragment thereof. In some embodiments, the compositions provided herein can include a mixture of an OspA polypeptide or an antigenic fragment thereof, a DbpA polypeptide or an antigenic fragment thereof, an OspC polypeptide or an antigenic fragment thereof, a P100 polypeptide or an antigenic fragment thereof, and a VlsE polypeptide or an antigenic fragment thereof.

In some embodiments, the compositions provided herein can include a mixture of a NapA polypeptide or an antigenic fragment thereof, an OspA polypeptide or an antigenic fragment thereof, an OspC polypeptide or an antigenic fragment thereof, and a Vlse polypeptide or an antigenic fragment thereof.

In some embodiments, the compositions provided herein can include a mixture of a NapA polypeptide or an antigenic fragment thereof, an OspA polypeptide or an antigenic fragment thereof, a DbpA polypeptide or an antigenic fragment thereof, an OspC polypeptide or an antigenic fragment thereof, a P100 polypeptide or an antigenic fragment thereof, and a Vlse polypeptide or an antigenic fragment thereof.

In some embodiments, the compositions provided herein can include an OspC polypeptide comprising an amino acid sequence having at least 80% (e.g., 80%, 85%, 90%, 95%, 100%) sequence identity to any one of SEQ ID NOs:1-4, or to an antigenic fragment thereof. In some embodiments, the compositions provided herein can include an OspC polypeptide having an amino acid sequence of any one of SEQ ID NOs:1-4. In some embodiments, the compositions provided herein can include an OspC polypeptide having an amino acid sequence of SEQ ID NO:1.

In some embodiments, the compositions provided herein can include a P100 polypeptide comprising an amino acid sequence having at least 80% (e.g., 80%, 85%, 90%, 95%, 100%) sequence identity to SEQ ID NOs:5, or to an antigenic fragment thereof. In some embodiments, the compositions provided herein can include an P100 polypeptide having an amino acid sequence of SEQ ID NOs:5.

In some embodiments, the compositions provided herein can include a VlsE polypeptide comprising an amino acid sequence having at least 80% (e.g., 80%, 85%, 90%, 95%, 100%) sequence identity to any one of SEQ ID NOs:6-7, or to an antigenic fragment thereof. In some embodiments, the compositions provided herein can include a VlsE polypeptide having an amino acid sequence of any one of SEQ ID NOs:6-7. In some embodiments, the compositions provided herein can include a VlsE polypeptide having an amino acid sequence of SEQ ID NO:6.

In some embodiments, the compositions provided herein can include a DbpA polypeptide comprising an amino acid sequence having at least 80% (e.g., 80%, 85%, 90%, 95%, 100%) sequence identity to any one of SEQ ID NOs:8-10, or to an antigenic fragment thereof. In some embodiments, the compositions provided herein can include a DbpA polypeptide having an amino acid sequence of any one of SEQ ID NOs:8-10. In some embodiments, the compositions provided herein can include a DbpA polypeptide having an amino acid sequence of SEQ ID NO:8.

In some embodiments, the compositions provided herein can include a DbpB polypeptide comprising an amino acid sequence having at least 80% (e.g., 80%, 85%, 90%, 95%, 100%) sequence identity to any one of SEQ ID NOs:11-13, or to an antigenic fragment thereof. In some embodiments, the compositions provided herein can include a DbpB polypeptide having an amino acid sequence of any one of SEQ ID NOs: 11-13. In some embodiments, the compositions provided herein can include a DbpB polypeptide having an amino acid sequence of SEQ ID NO:11.

In some embodiments, the compositions provided herein can include a NapA polypeptide comprising an amino acid sequence having at least 80% (e.g., 80%, 85%, 90%, 95%, 100%) sequence identity to any one of SEQ ID NOs:14-15, or to an antigenic fragment thereof. In some embodiments, the compositions provided herein can include a NapA polypeptide having an amino acid sequence of any one of SEQ ID NOs:14-15. In some embodiments, the compositions provided herein can include a NapA polypeptide having an amino acid sequence of SEQ ID NO:14.

In some embodiments, the compositions provided herein can include an OspA polypeptide comprising an amino acid sequence having at least 80% (e.g., 80%, 85%, 90%, 95%, 100%) sequence identity to any one of SEQ ID NOs:16-18, or to an antigenic fragment thereof. In some embodiments, the compositions provided herein can include a NapA polypeptide having an amino acid sequence of any one of SEQ ID NOs:16-18. In some embodiments, the compositions provided herein can include a NapA polypeptide having an amino acid sequence of SEQ ID NO:16.

In some embodiments, the compositions provided herein can include a P41 polypeptide comprising an amino acid sequence having at least 80% (e.g., 80%, 85%, 90%, 95%, 100%) sequence identity to any one of SEQ ID NOs:19-21, or to an antigenic fragment thereof. In some embodiments, the compositions provided herein can include a P41 polypeptide having an amino acid sequence of any one of SEQ ID NOs:19-20. In some embodiments, the compositions provided herein can include a P41 polypeptide having an amino acid sequence of SEQ ID NO:19.

In some embodiments, the compositions provided herein can include a BmpA polypeptide comprising an amino acid sequence having at least 80% (e.g., 80%, 85%, 90%, 95%, 100%) sequence identity to any one of SEQ ID NOs:21-23, or to an antigenic fragment thereof. In some embodiments, the compositions provided herein can include a BmpA polypeptide having an amino acid sequence of any one of SEQ ID NOs:21-23. In some embodiments, the compositions provided herein can include a BmpA polypeptide having an amino acid sequence of SEQ ID NO:21.

Concurrent infections of Lyme disease and other tick-borne illnesses can occur. Thus, a composition provided herein can include one or more antigens derived from or exhibiting sequence similarity to one or more tick-borne infectious agents. For example, a composition can include one or more polypeptides derived from a species of the protozoan parasite Babesia (e.g., Babesia bovis, Babesia divergens, Babesia microti). A composition can include one or more polypeptides derived from a species of the Gram-negative bacterium Bartonella (e.g., Bartonella bacilliformis, Bartonella henselae, Bartonella quintana, Bartonella rochalimae), from a species of the rickettsiales bacteria genus Anaplasma (e.g., Anaplasma phagocytophilum) or the genus Ehrlichia (e.g., Ehrlichia ewingii, Ehrlichia chaffeenis, Ehrlichia canis, Neorickettsia sennetsu), from a species of mycoplasma bacteria (e.g., Mycoplasma fermentans, Mycoplasma hominis, Mycoplasma pneumoniae, Mycoplasma genitalium, Mycoplasma penetrans), or from a species of Rickettsia (e.g., Rickettsia rickettsii, Rickettsia typhi) and others.

### Methods For Detecting Cytokines and Diagnosing Lyme Disease

Cytokines are the chemical messengers of the immune system and serve as markers for inflammatory processes. An encounter with physical stimuli (i.e. bacterial, viral, fungal, parasite infection, toxins, food, allergens, auto antigens, or medications) challenges the immune system. In response to such stimuli, immune cells secrete cytokines as a primary defense mechanism. Elevated cytokine levels may be an indication of an active immune response to physical stimuli.

Cytokine assays for determining immunological responsiveness generally involve measuring cytokine or growth factor production. Cytokine assays can be performed using peripheral blood mononuclear cells (PBMCs). PBMCs can be obtained from a subject's whole blood. Suitable methods for obtaining PBMCs can be used. For example, a whole peripheral blood sample can be obtained from a subject having or suspected of having Lyme disease (e.g., experiencing symptoms associated with Lyme disease). The PBMCs, which include lymphocytes, macrophages, and other white blood cells, can be isolated from whole peripheral blood by any appropriate method (e.g., centrifugation or density gradient). In some embodiments, PBMCs can be isolated from the whole blood by centrifugation, washed, and then suspended in medium with antibiotic, e.g., RPMI medium (Gibco, Grand Island, N.Y.) with penicillin/streptomycin and 1% glutamine.

PBMCs can be cultured alone or in the presence of a composition provided herein (e.g., Lyme antigens) or a control antigen (e.g., phytohemagglutinin). Cell-free supernatants can be collected from stimulated and non-stimulated or control cell cultures for cytokine analysis. Among the factors that can be measured using cytokine assays are: any of the interleukins (IL), tumor necrosis factors (TNF), interferons (IFN), colony stimulating factors (CSF), leukemia inhibitory factor (LIF), transforming growth factors (TGF), or epidermal growth factor (EGF). In some embodiments, levels of one or more cytokines such as IL-2, IL-3, IL-4, IL-5, IL-6, IL-9, IL-10, IL-13, IL-17A/F, IL-21, IL-22, IL-25, IL-31, TNF-α, TNF-β, IFN-γ, and GM-CSF, can be measured. Methods of measuring one or more cytokine levels can include, for example, a bioassay, an immunoassay, a radioimmunoassay (RIA), an enzyme-linked immunosorbent assay (ELISA), an enzyme-linked immunosorbent spot assay (ELISpot), or measurement of messenger RNA levels. In general, immunoassays involve using a monoclonal antibody to the cytokine of interest to specifically bind and detect the cytokine. Immunoassays are well-known in the art and can include both competitive assays and immunometric assays (see Ausubel et al., Current Protocols in Molecular Biology, 11.2.1-11.2.19 (1993); Laboratory Techniques in Biochemistry and Molecular Biology, Work et al., ed. (1978)).

The enzyme-linked immunosorbent spot (ELISpot) assay allows visualization of a secretory product of individual activated or responding cells. Each spot developed in the assay represents a single reactive cell. Thus, the ELISpot assay can accurately detect, measure, and perform functional analysis of low-frequency immune cells at single cell resolution.

The immune responses to a Lyme-specific Borrelia antigen include both B cell and T cell activation. T cell-mediated cytokine secretion occurs much earlier in the disease progression than B cell-mediated antibody response. Assessment of both the function and the frequency of Borrelia-specific T cells can help evaluate the cellular immune response to, and diagnosis of Borrelia infection (Dressler, F., et al., Ann. Intern. Med. 115: 533-539, 1991; Gross, D. M., et al., Science 281: 703-706, 1998). Due to the clonal expansion (proliferation) of antigen-specific T cells in vivo during an immune response, the presence of increased frequencies of Borrelia antigen-specific effector/memory T cells in peripheral blood suggests prior infection/exposure to Borrelia (Widhe, M., et. al., J. Infect. Dis. 189: 1881-1891, 2004; Widhe, M., et al., Int. Immunol. 17: 1283-1291, 2005). As disclosed herein, a novel highly sensitive Lyme disease-specific ELISpot assay (Lyme ELISpot) was developed to detect cytokine secretion by T lymphocytes that have been pre-sensitized in vivo to Lyme-specific Borrelia antigens. High sensitivity of the Lyme ELISpot assay is ensured by a mixture of serum-free medium, purified recombinant Lyme antigens, and/or co-stimulation by Interleukin-7 (IL-7).

Lyme ELISpot assays can be performed using peripheral blood mononuclear cells (PBMCs). PBMCs can be obtained from a subject's whole blood using suitable methods (e.g., centrifugation or density gradient). In some embodiments, PBMCs can be isolated from the whole blood by centrifugation, washed, and then suspended in serum-free medium with antibiotic, e.g., CTL-Test Medium (Cellular Technology Limited).

To detect pre-sensitized T lymphocytes, Lyme ELISpot assays can employ two high-affinity antibodies directed against different epitopes on the same secreted cytokine molecule. More specifically, a microtiter plate can be coated with a capture antibody specific to a first epitope on the secreted cytokine PBMCs can then be plated on the microtiter plate at a desired density in serum-free medium alone (negative control), or in serum-free medium with the addition of one or more Lyme antigens or PHA (positive control), and incubated in a humidified 37° C. CO₂ incubator for a specified period of time. In some embodiments, IL-7 can be added to the serum-free medium and used for all conditions. The incubation time can be sufficient to permit T-lymphocytes that have been pre-sensitized to Lyme antigens to secrete cytokines, for example the incubation time could be 18-72 hours, depending on the specific cytokine studied, preferably, 18-24 hours for IFN-γ and IL-2, 36-48 hours for IL-4 and IL-17. During the incubation period, the cytokines secreted by T-lymphocytes are captured locally by the capture antibodies immobilized on the plate.

After washing the wells to remove cells, debris, and media components, a detection antibody specific for a distinct second epitope on the cytokine can be added to detect the captured cytokines. The detection antibody can be any detectably labeled antibody, for example, an antibody tagged with a fluorescent dye, an enzyme-conjugated antibody, or an antibody conjugated with one member of a specific binding pair, e.g., an antibody conjugated with biotin or streptavidin. For example, when a biotinylated detection antibody is employed, enzyme-conjugated streptavidins can also be used to detect the cytokine-antibody complexes. After washing to remove any unbound detection antibody, the captured cytokine-antibody complexes can then be visualized using a chromogenic, fluorogenic, or electrochemiluminescent substrate of the enzyme. Because the cytokines are captured locally upon secretion, each visible spot represents an individual cytokine-secreting T cell, offering single cell resolution for the test. The spots can be counted manually (e.g., with a dissecting microscope) or using an automated analyzer, e.g., CTL-ImmunoSpot Analyzer (Cellular Technology Limited).

Lyme ELISpot assays can be used to diagnose Lyme disease in a subject, e.g., a human being or a domesticated animal. Such a method can include the following steps: (a) providing peripheral blood mononuclear cells of the subject; (b) incubating the peripheral blood mononuclear cells in a serum-free medium with one or more Lyme antigens and interleukin-7; and (c) measuring one or more cytokines secreted by the peripheral blood mononuclear cells, wherein an increase of the one or more cytokines relative to a control is indicative of Lyme disease in the subject. Cytokines that can be measured in Lyme ELISpot assays include IL-2, IL-3, IL-4, IL-5, IL-6, IL-9, IL-10, IL-13, IL-17A/F, IL-21, IL-22, IL-25, IL-31, TNF-α, TNF-β, IFN-γ, and GM-CSF.

Any composition of Lyme antigens described herein can be used for the Lyme ELISpot assay. Titration experiments can be performed to determine suitable concentrations for the Lyme antigens. While the traditional two-tiered ELISA and Western Blot tests detect the serological presence of antibodies to Lyme antigens, the Lyme ELISpot tests detect Lyme antigen-activated cytokine secretion by T lymphocytes, which occur earlier in disease progression. Thus Lyme ELISpot assay is capable of early diagnosis of Lyme disease, and can detect antigen-specific T cell responses in seronegative Lyme patients as well.

In some embodiments, Lyme ELISpot assays can be used in parallel with other methods of diagnosing Lyme disease, including subjective (e.g., self-report of symptoms) and objective measurements of Lyme disease symptoms. For example, the methods provided herein can be used in parallel with clinical observations of, or a subject's self-reporting of, tick bite, erythema migrans (or bull-eye shaped rash), skin lesion, pain, fever, headache, swelling, or other symptoms associated with Lyme disease. In some embodiments, Lyme ELISpot assays can be used in parallel or mixture with Western blot analysis or serological assays for the presence of Borrelia-specific antibodies.

In some embodiments, Lyme ELISpot assays can be used in mixture with other assays to differentiate active ongoing Borrelia infection from a previous or latent Borrelia infection. For example, Lyme ELISpot assays can be used in mixture with IL-17 ELISpot, Granzyme B or Perforin ELISpot, B cell ELISpot, Bio-Plex Pro human Th17 cytokine assays (BIO-RAD), and/or flow cytometry-based detection of Borrelia-specific T cells by using Borrelia peptide-MHC Dextramer (IMMUDEX), to determine the Lyme disease stage (Oosting et al., Eur. J. Immunol. 41: 172-181, 2011; Henningsson et al., Journal of Neuroinflammation 8:36, 2011; Codolo et al., Arthritis & Rheumatism 58 (11): 3609-3617, 2008).

### Methods For Treating and Monitoring Treatment of Lyme Disease

Also provided herein are methods of treating Lyme disease in a subject. Such methods include (1) providing peripheral blood mononuclear cells (PBMCs) of the subject; (2) incubating the PBMCs in a serum-free medium with one or more Lyme antigens; (3) measuring the level of one or more cytokines secreted by the PBMCs; and (4) if the level of the one or more cytokines is above a control level, administering to the subject a treatment comprising one or more antibiotics. The control level can be the level of one or more cytokines secreted by the PBMCs in a healthy subject.

Treatments for Lyme disease include, without limitation, administration of antibiotics, e.g., doxycycline, amoxicillin, cefuroxime axetil, ceftriaxone, cefotaxime, penicillin, and azithromycin, either orally or intravenously. For some subjects with early stage of active Borrelia infection, treatment with antibiotics for a normal period (e.g., a week) can cure the infection completely. Some patients, particularly those diagnosed with later stages of Lyme disease, may have persistent or recurrent symptoms. For such subjects, extended antibiotic therapy (e.g., one, two, three, four, or more weeks of antibiotic treatment) can be administered.

As used herein, the term “treat” or “treatment” is defined as the application or administration of a treatment regimen, e.g., a therapeutic agent or modality, to a subject, e.g., a patient. The subject can be a patient having Lyme disease or a symptom of Lyme disease, or at risk of developing Lyme disease (e.g., frequently outdoors, living in a tick infested area). The treatment can be to cure, heal, alleviate, relieve, alter, remedy, ameliorate, palliate, improve or affect Lyme disease or symptoms associated with Lyme disease.

Also provided herein are methods of monitoring a subject's response to the Lyme disease treatment. For example, the methods provided herein can be used to monitor a subject's response to standard therapeutic regimen for Lyme disease, which include administration of one or more of the above described antibiotics. In some embodiments, the methods provided herein also can be used to monitor a subject's response to standard therapeutic regimen for a co-infection of Babesia, Bartonella, Ehrlichia, Anaplasma, and/or Rickettsia. Standard therapeutic regimens for Babesia can include administration of medicaments such as atovaquone (Mepron) plus azithromycin (Zithromax), clindamycin and oral quinine. Standard therapeutic regimens for Bartonella can include administration of medicaments such as erythromycin, fluoroquinolone, or rifampin. Ehrlichia is frequently treated with the administration of medicaments such as doxycycline and rifampin.

Lyme ELISpot assays can be used to monitor a subject's response to Lyme disease treatment by, for example, determining a level of one or more cytokines at multiple time-points. The subject can be monitored in one or more of the following periods: before the treatment; during the treatment; or after one or more elements of the treatment have been administered. For example, Lyme ELISpot assays can be performed at predetermined time-point before or while a subject is treated with antibiotics. Lyme ELISpot assays can be repeated at one or additional later time-points to observe any change in the levels of one or more cytokines over time.

In some embodiments, a first sample of peripheral blood mononuclear cells (PBMC) can be obtained from a subject prior to receiving a treatment, and a first level of one or more cytokines secreted by the first sample of PBMC can be measured by Lyme ELISpot assay. A second sample of PBMC can be obtained from the subject during or after a treatment of Lyme disease, and a second level of one or more cytokines secreted by the second sample of PBMC can be measured by Lyme ELISpot assay. The first level and the second level can then be compared to assess the effectiveness of the treatment. If the second level is lower than the first level, the treatment may be effective for the subject. Conversely, if the second level is higher than or similar to the first level, the treatment may be ineffective for the subject. Monitoring can be used to evaluate the need for further treatment with the same or a different therapeutic agent or modality.

In some embodiments, Lyme ELISpot assays can be used in parallel with other methods of monitoring Lyme disease treatment, including subjective (e.g., self-report of symptoms) and objective measurements of Lyme disease symptoms. For example, the methods provided herein can be used in parallel with clinical observations of, or a subject's self-reporting of, tick bite, erythema migrans (or bull-eye shaped rash), skin lesion, pain, fever, headache, swelling, or other symptoms associated with Lyme disease. In some embodiments, Lyme ELISpot assays can be used in parallel with Western blot analysis or serological assays for the presence of Borrelia-specific antibodies.

### Kits

Also provided herein are kits including one or more of the compositions provided herein. Instructions for use can include instructions for diagnostic applications of the compositions for diagnosing Lyme disease and/or monitoring the response of a subject to treatment of Lyme disease. The kit can include one or more other elements including: instructions for use and other reagents such as serum-free medium; IL-7; a positive control, e.g., phytohaemagglutinin (PHA); microtiter plates coated with one or more capture antibodies specific to a cytokine, e.g., IFN-γ; a detection antibody to the cytokine, e.g., IFN-γ; a substrate; buffers and antimicrobial agents. The capture and detection antibodies can be monoclonal or polyclonal antibodies that bind to different epitopes on the cytokine. The detection antibody can be any detectably labeled antibody, for example, an antibody tagged with a fluorescent dye, e.g., an Alexa Fluor 488-conjugated antibody; an enzyme-conjugated antibody, e.g., alkaline phosphatase-conjugated antibody; or an antibody conjugated with one member of a specific binding pair, e.g., an antibody conjugated with biotin or streptavidin. For example, when a biotinylated detection antibody is included in the kit, the kit also includes enzyme-conjugated streptavidin, e.g., alkaline phosphatase-conjugated streptavidin. The kit can include a chromogenic, fluorogenic, or electrochemiluminescent substrate of the enzyme on the detection antibody or strepavidin. For example, a chromogenic substrate for alkaline phosphatase can be a 5-Bromo-4-chloro-3-indolyl phosphate (BCIP), nitro blue tetrazolium chloride (NBT), or a mixture of BCIP and NBT. The instructions for use can be in a paper format or on a CD or DVD.

Kits as provided herein can be used in accordance with any of the methods described above, e.g., diagnosing or monitoring Lyme disease. Those skilled in the art will be aware of other suitable uses for kits provided herein, and will be able to employ the kits for such uses. Kits as provided herein can also include a mailer (e.g., a postage paid envelope or mailing pack) that can be used to return the sample for analysis, e.g., to a laboratory. The kit can include one or more containers for the sample, or the sample can be in a standard blood collection vial. The kit can also include one or more of an informed consent form, a test requisition form, and instructions on how to use the kit in a method described herein. Methods for using such kits are also included herein. One or more of the forms (e.g., the test requisition form) and the container holding the sample can be coded, for example, with a bar code for identifying the subject who provided the sample.

### EXAMPLES

The invention is further described in the following examples, which do not limit the scope of the invention described in the claims.

### Example 1

### Lyme ELISpot Assay

Both humoral and cellular immune responses develop in Borrelia infection. Assessment of both the function and the frequency of Borrelia-specific T cells can help evaluate the cellular immune response to, and diagnosis of Borrelia infection (Dressler, F., et al., Ann. Intern. Med. 115: 533-539, 1991; Gross, D. M., et al., Science 281: 703-706, 1998). Due to the clonal expansion (proliferation) of antigen-specific T cells in vivo during an immune response, the presence of increased frequencies of Borrelia antigen-specific effector/memory T cells in peripheral blood suggests prior infection/exposure to Borrelia (Widhe, M., et. al., J. Infect. Dis. 189: 1881-1891, 2004; Widhe, M., et al., Int. Immunol. 17: 1283-1291, 2005).

The frequency of Borrelia-specific effector/memory T cells in PBMC was studied by performing Lyme ELISpot assays to measure the numbers of T cells that secreted IFN-γ upon ex vivo stimulation by Lyme antigens. Human peripheral blood mononuclear cells (PBMCs) were isolated from whole blood samples, obtained from a cohort of 80 healthy donors and 25 diagnosed Lyme patients. All individuals that were classified as Lyme patients met the CDC surveillance definition of Lyme disease, including clinical signs and symptoms, history of possible exposure to infected blacklegged ticks, with or without a positive antibody response to Borrelia burgdorferi by ELISA and Western Blot, interpreted according to CDC and the Infectious Disease Society of America (IDSA) criteria.

The PBMCs were plated at 250,000 cells per well in anti-IFN-γ antibody pre-coated 96-well plates (part of human IFN-γ ELISPOT kit by Cellular Technology Limited, Ohio, USA). The PBMCs were then stimulated with recombinant Lyme antigens in serum-free medium, in the presence or absence of Interleukin-7 (1.3 ng/ml, R&D Systems, Minn., USA). Serum-free medium alone served as a negative control, and phytohaemagglutinin (PHA), a known activator of T-lymphocytes, was used as positive control. All Borrelia antigens were purchased from DIARECT AG (Freiberg, Germany). All culture conditions (negative control, positive control, and Lyme antigen stimulation) were tested in triplicate. PBMCs were incubated for 18-24 hours at 37° C., 9% CO₂. During the incubation period, IFN-γ secreted by T-lymphocytes was captured by the anti-IFN-γ antibody immobilized on PVDF membranes. After incubation, the wells were washed with serum-free medium to remove any unbound material. A biotinylated anti-IFN-γ antibody directed against a different epitope on IFN-γ (part of human IFN-γ ELISPOT kit by Cellular Technology Limited, Ohio, USA), was added to the wells and allowed to react for 2 hours. The wells were washed again, and streptavidin conjugated alkaline-phosphatase (Strep-AP) were added to each well. After unbound Strep-AP was removed by washing, a mixture of 5-Bromo-4-chloro-3-indolyl phosphate and nitro blue tetrazolium chloride was added to the wells. A blue-colored precipitate formed and appeared as a spot at each IFN-γ-antibody complex. The wells are washed again and dried overnight prior to plate analysis. The results of ELISPOT were analyzed using the CTL S6 Ultimate-V Analyzer/BioSpot 5.0 Software (CTL, Ohio, USA) and reported as IFN-γ Spot Forming Units (SFU). Parameters for the analyzer were set to be: spot separation=1, diffuse processing=Large, adjusted count area=95%, fiber removal=yes. The number of spot forming units (SFU) was counted by an automated ImmunoSpot reader. The number of spots represents the amount of cytokines secreted by T lymphocytes.

In some assays, the Lyme antigens used was Antigen mix 1, a mixture of a recombinant OspC polypeptide having an amino acid sequences of SEQ ID NO:1, a recombinant P100 polypeptide having an amino acid sequences of SEQ ID NO:5, a recombinant VlsE polypeptide having an amino acid sequences of SEQ ID NO:6, and a recombinant DbpA polypeptide having an amino acid sequences of SEQ ID NO:8. In some assays, the Lyme antigens used was Antigen mix 2, a mixture of a recombinant OspC polypeptide having an amino acid sequences of SEQ ID NO:1 and a recombinant VlsE polypeptide having an amino acid sequences of SEQ ID NO:6. In some assays, the Lyme antigen used was a recombinant NapA polypeptide having an amino acid sequences of SEQ ID NO:14. In some assays, the Lyme antigen used was a recombinant OspA polypeptide having an amino acid sequences of SEQ ID NO:16.

Titration experiments were performed to determine suitable concentrations for the Lyme antigens. A final concentration of 12.5 ug/ml NapA provided good results for Lyme ELISpot assays (FIG. 1); and a final concentration of 12.5 ug/ml OspA also provided good results (FIG. 2). The preferable final concentrations for the other Lyme antigens were determined to be: 6 μg/ml for DbpA, 12.5 μg/ml for OspC, 32.5 μg/ml for P100, and 12.5 μg/ml for VlsE (data not shown).

A representative Lyme ELISpot report of a Lyme antigen negative donor was shown in FIG. 3. The Lyme antigen mixtures used were: Antigen mix 1 and Antigen mix 2. Serum-free medium alone serves as a negative control, and PHA (phytohaemagglutinin) serves as a positive control. The results were reported as the absolute IFN-γ spot forming units (SFU) per well of 250,000 PBMC cells. Both the Lyme ELISpot assay with IL-7 and the Lyme ELISpot assay without IL-7 reported negative results (FIG. 3).

Representative Lyme ELISpot reports of Lyme antigen positive patients were shown in FIGS. 4-5. The Lyme antigen mixtures used were: Antigen mix 1 and Antigen mix 2. For some Lyme patients, both the Lyme ELISpot assay with IL-7 and the Lyme ELISpot assay without IL-7 reported positive results, and the addition of IL-7 dramatically amplified the signal of IFN-γ-forming spots (FIG. 4). For some Lyme patients, however, Lyme ELISpot assay with IL-7 and the Lyme ELISpot assay without IL-7 reported different results: while Lyme ELISpot assay without IL-7 reported negative results, Lyme ELISpot assay with IL-7 reported positive results (FIG. 5). These findings indicated that the addition of IL-7 improved the detection of T lymphocytes that have been pre-sensitized to Lyme antigens, without increasing non-specific spots in healthy controls and the medium control background.

Statistical analysis of the results of Lyme ELISpot assay with IL-7 and those of Lyme ELISpot assay without IL-7 were performed on eight to ten Lyme patients. The addition of IL-7 significantly (P=0.017) improved the detection of Lyme antigen pre-sensitized T cells (FIG. 6A and 6B).

FIG. 7A shows the Lyme ELISpot assay using Antigen mix 1 and IL-7 (“iSpot Lyme assay”) significantly increased (p=0.001) the sensitivity for detecting Borrelia-reactive T cells when compared to the Lyme ELISpot assay using Antigen mix 2 but not IL-7. The spot size distribution was analyzed and compared between these two Lyme ELISpot assays. FIG. 7B shows that the spot sizes in both Lyme ELISpot assays showed the normal distribution, which is characteristic of the cytokine signature of T cells. There was no difference in the spot size generated in the two assays (FIG. 7B). Therefore, these data suggest that the use of IL-7 and Antigen mix 1 in the “iSpot Lyme assay” specifically increases the number of Borrelia-reactive T cells that secrete IFN-γ, but does not change the amount of IFN-γ produced from each of these Borrelia-reactive T cells.

These results suggest that the Lyme ELISPOT assay, especially the “iSpot Lyme assay,” is a highly sensitive in vitro assay for detecting specific T cell immune response to Borrelia infection.

### Example 2

### Sensitivity and Specificity of the iSpot Lyme Assay as a Diagnostic Test for Borrelia Infection

The specificity and sensitivity of the “iSpot Lyme assay” (the Lyme ELISpot assay using Antigen mix 1 and IL-7) as a laboratory T cell-based diagnostic test for Borrelia infection were further investigated. For this purpose, PBMC were isolated from 80 healthy controls that had not been exposed to Borrelia (HC), 25 patients with clinically diagnosed Lyme disease (LD) and 23 non-Lyme patients (NLP) who had clinical symptoms similar to Lyme disease but did not actually have Lyme disease. As shown in FIG. 8A, the iSpot Lyme assay clearly distinguished the Lyme disease patients from healthy controls and non-Lyme patients, in both cases with a significance level of p<0.0001.

To further assess the performance of the iSpot Lyme assay, the Receiver Operating Characteristic (ROC) analysis was performed and used to determine the sensitivity, specificity, the positive predictive value (PPV) and the negative predictive value (NPV) of the iSpot Lyme assay. An ROC curve is a plot of test sensitivity (plotted on the y axis) versus its false positive rate (1-specificity) (plotted on the x axis). In this study, the iSpot Lyme assay showed a sensitivity of 84%, a specificity of 94%, a PPV of 81%, a NPV of 95%, and an area under the curve value (AUC) of 0.943(FIG. 8B). In contrast, the Lyme ELISpot assay using Antigen mix 2 but not IL-7 showed a sensitivity of 67%, a specificity of 76%, a PPV 48%, a NPV of 86%, and an area under the curve value (AUC) of 0.68 (FIG. 8C). The cutoff value as determined by ROC is 25 SFU per well for the iSpot Lyme assay.

Overall, the ROC analysis suggests that the iSpot Lyme assay fulfills the criteria of a reliable diagnostic laboratory test for Borrelia infection.

### Example 3

### Optimization and Validation of the iSpot Lyme Assay

To determine the reliability of the iSpot Lyme assay as a routine laboratory test, its intra- and inter-assay precision was studied. For the intra-assay precision studies, PBMC from five diagnosed Lyme patients were selected who displayed high, medium and low Lyme antigen-triggered SFU values. Each of the PBMC samples was run in triplicate. As shown in FIG. 9A, the coefficient of variation (CV) among the triplicates ranged from 4.6% to 18.1% with a trend showing that an increase in CV is inversely proportional to SFU values. Inter-assay precision measurements were performed on three diagnosed Lyme patient PBMC samples on five consecutive days. To make sure that identical cell material was tested and to avoid a biological variation of the sample itself due to blood collections at different times, cryopreserved PBMC samples from a single blood draw were used for the assays (one aliquot thawed each day). The coefficient of variation was 5.4%, 5.9% and 13.1% for the three donors (FIG. 9B). These data suggested that the iSpot Lyme assay is a reliable test in terms of intra-assay and inter-assay precision.

The results of an ELISpot assay can be influenced by the PBMC numbers plated. Therefore, the relationship between the Lyme antigen-induced IFN-γ SFU value and the PBMC numbers plated per well was studied. A linear relationship was observed between the Lyme antigen-induced IFN-γ SFU value and the PBMC numbers per well (FIG. 9C). These results are similar to observations made in other antigen-specific ELISpot systems (Zhang, W., et al., J. Immunotoxicol. 6: 227-234, 2009). These data show that the variability of Lyme antigen-induced SFU value depends on the accuracy of PBMC cell count when adjusting the PBMC concentration, and the precision of pipetting the cells. With 250,000 PBMC plated per well, the variability is directly proportional to the magnitude of such imprecisions. When PBMC of healthy controls were plated in increasing numbers, the IFN-γ spot numbers did not increase with or without Lyme antigen in the test system (FIG. 9D), suggesting that the IFN-γ spots are produced specifically by Borrelia-reactive T cells in response to ex vivo restimulation of Lyme antigen.

Antigen concentration can affect activation of the antigen-specific T cells (Hesse, M. D., et al., J. Immunol. 167: 1353-1361, 2001). The Lyme antigen mix 1 or 2 was diluted in serial dilutions and tested in Lyme ELISpot assays performed on Borrelia positive donors. As shown in FIG. 9E, when the Lyme antigen concentration was increased, the value of SFU initially increased rapidly but reached a plateau when the Lyme antigen concentration is at or over 10 μg/mL. The Lyme antigen dose-response curve was very similar for the iSpot Lyme assay and the Lyme ELISpot assay using Antigen mix 2 but not IL-7. The SFU values were significantly higher in the iSpot Lyme assay when compared to the Lyme ELISpot assay using Antigen mix 2 but not IL-7 (FIG. 9E). Therefore, a Lyme antigen mix concentration of 10 μg/mL is in the plateau of the dose response curve and any inaccuracy occurred in pipetting the Lyme antigen mix at this concentration is a low risk factor for affecting the test result. These results confirm that the iSpot Lyme assay is suitable for detecting the low frequency Borrelia-specific effector/memory T cells.

Assay validation often includes determination of accuracy by an independent readout system (Fardy, J. Evaluation of Diagnositc Tests. In Methods of Molecular Biology, Clinical Epidemiology; Parfrey, P., Barrett, B., Eds.; Humana Press: Totowa, N.J., USA, 2009; Volume 473, pp. 127-136). The concentrations of IFN-γ in the supernatant from Lyme antigen stimulated PBMC were determined using the Bio-Plex suspension array system according to the manufacturer's instructions (Bio-Rad, Hercules, Calif., USA), and used as a validation system for Lyme ELISpot assays. Briefly, supernatants were collected from 96-well plates containing PBMC that were stimulated overnight with Lyme antigens, and frozen at −80 ° C. until use. The thawed supernatant samples were incubated in 96-well filter plates at room temperature for 30 min with antibodies chemically coupled to fluorescent-labeled microbeads. After three washes, premixed detection antibodies were added to each well and incubated for 30 min. Following three washes, premixed streptavidin-phycoerythrin was added to each well and incubated for 10 min. Finally, the beads were washed three times and resuspended with 125 μL of assay buffer. The plates were read on a Bio-Plex 200 reader and data were processed and analyzed by using Bio-Plex Manager Software 6.0 (Bio-Rad, Hercules, Calif., USA). Values with coefficient of variation (% CV) above 30 were excluded from the standard curve.

The correlation between IFN-γ SFU numbers as established by the iSpot Lyme assay and the concentration of soluble IFN-γ in the culture supernatants as measured by the Bio-Plex method was determined. As shown in FIG. 9F, the results of the iSpot Lyme assay were closely correlated to the IFN-γ concentrations as measured by the Bio-Plex method (R=0.81 and p<0.0001).

Overall, the validation results showed that the iSpot Lyme assay is a reliable and sensitive test for detecting Borrelia-specific T cells with the potential application in clinical laboratory diagnosis for Borrelia infection and Lyme disease.

### Example 4

### Comparison of Lyme ELISpot Assay With Western Blot

Conventionally, the Western Blot assay was used in the clinical diagnosis of Lyme disease (Aguero-Rosenfeld, M. E., et. al., Clin. Microbiol. Rev. 18: 484-509, 2005). Lyme patients can be classified into two groups according to their serum antibody reactivity to Borrelia antigens (Dattwyler, R. J., et. al., N. Engl. J. Med. 319: 1441-1446, 1988). Patients who are Lyme positive as identified by the Western Blot are called “seropositive Lyme patients;” and patients who do not have detectable antibody levels are defined as seronegative patients. Sensitivity of Western blot, Lyme ELISpot assay using antigen mix 2 but not IL-7 and the iSpot Lyme assay was compared and analyzed. Lyme ELISpot assays were performed as described in Example 1. Western blot analyses were performed on patient serum samples by using Borrelia Western Blot IgG and IgM kits (Trinity Biotech, Carlsbad, Calif., USA) following the manufacturer's instruction. Aliquots (20 μl) of undiluted serum samples were added to channels containing the test strips and 2 ml of dilution buffer. Antigens on membranes of this kit were separated by the manufacturer and IgG kit includes the following 13 bands: p18, p23, p28, p30, p31, p34, p39, p41, p45, p58, p60, p66, and p93; IgM kit includes p23, p39, and p14. The strips were scanned using BLOTrix Reader (Frankfurt, Germany). Visualization of specific protein bands indicated the presence of serum IgG or IgM antibodies against B. burgdorferi-derived antigens. Samples were classified as positive or negative in accordance with the criteria established by CDC.

In a study of 23 diagnosed Lyme patients, including both seropositive and seronegative patients, the Western Blot assay was able to identify 30% of these Lyme patients as positive; the iSpot Lyme assay was able to identify 84% of these Lyme patients as positive, whereas the Lyme ELISpot using Antigen mix 2 but not IL-7 was able to identify 50% of them as positive (FIG. 10A). In a study of the 17 seronegative Lyme patients, the Western blot failed to identify any Lyme positive patients among them; the Lyme ELISpot assay without using IL-7 was able to identify 47% of the seronegative Lyme patients as positive; and the iSpot Lyme assay was able to identify 82% of them as positive (FIG. 10B). Therefore, the Lyme ELISpot assays with or without using IL-7 provided an overall greater sensitivity in detecting Lyme patients when compared to conventional Western blot (FIG. 10A-B). While the Western blot reported false negative results for all 17 seronegative Lyme patients, the Lyme ELISpot assay using antigen mix2 but not IL-7 correctly diagnosed 47% of the seronegative Lyme patients and the iSpot Lyme assay correctly diagnosed 82% of those patients. The iSpot Lyme assay provides significantly higher sensitivity than the Lyme ELISpot assays using antigen mix 2 but not IL-7 and thus is particularly useful in diagnosing, treating and monitoring Lyme disease for seronegative Lyme patients.

In summary, these results demonstrate that the Lyme ELISpot assay is superior to the Western Blot assay in terms of sensitivity for detecting the underlying Borrelia infection. The data further suggest that the magnitudes of the humoral immune response and the T cell-mediated cellular immune response in Borrelia infection are dissociated. The data also illustrate that the iSpot Lyme assay can identify some Borrelia infected individuals when the serology-based diagnostic assay fails to do so.

To test the cross-reactivity of the Western Blot assay, the Lyme ELISpot without IL-7, and the iSpot Lyme assay, 23 patients who were from low risk areas of Borrelia infection and had other clinical conditions but not Lyme disease were studied. As shown in FIG. 10C, the Western Blot assay gave 36% false positive results whereas both the Lyme ELISpot without IL-7 and the iSpot Lyme assay did not show any cross-reactivity in those non-Lyme patients. Therefore, Lyme ELISpot assays, in particular the iSpot Lyme assay, are not only more sensitive but also more specific than the standard Western Blot serodiagnostic test for identifying the Lyme disease and Borrelia infection. Due to the apparent prevalence of both humoral and cellular immune responses in infected individuals, it is conceivable that the combination of the Lyme ELISpot assay with Western Blot assay would further increase the sensitivity of diagnosing the Lyme disease.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for diagnosing Lyme disease in a subject, the method comprising:
a. providing peripheral blood mononuclear cells (PBMCs) of the subject;
b. incubating the PBMCs in a serum-free medium with one or more Lyme antigens; and
c. measuring the level of one or more cytokines secreted by the PBMCs, wherein the level of one or more cytokines above a control level is indicative of Lyme disease in the subject.

2. The method of claim 1, wherein the serum-free medium contains interleukin-7.

3. The method of claim 1, wherein measuring the level of one or more cytokines comprises performing a bioassay, an immunoassay, a flow cytometry, or a radioimmunoassay (RIA).

4. The method of claim 3, wherein the immunoassay is an enzyme-linked immunosorbent spot (ELISpot) assay.

5. The method of claim 1, wherein the one or more cytokines are selected from the group consisting of IL-2, IL-3, IL-4, IL-5, IL-6, IL-9, IL-10, IL-13, IL-17A/F, IL-21, IL-22, IL-25, IL-31, TNF-α, TNF-β, IFN-γ, and GM-CSF.

6. The method of claim 5, wherein the cytokine is IFN-γ.

7. The method of claim 1, wherein the control level is the level of the one or more cytokines secreted by the peripheral blood mononuclear cells in a healthy subject.

8. The method of claim 1, wherein the one or more Lyme antigens are polypeptides or proteins derived from or exhibiting sequence similarity to polypeptides or proteins derived from one or more pathogenic species of Borrelia.

9. The method of claim 8, wherein the one or more pathogenic species of Borrelia is selected from the group consisting of Borrelia burgdorferi sensu stricto, Borrelia afzelii, Borrelia garinii, Borrelia valaisiana, Borrelia bissettii, Borrelia lusitaniae, and Borrelia spielmanii.

10. The method of claim 8, wherein the one or more Lyme antigens are selected from the group consisting of but not limited to a Variable major protein-like gene E (VlsE) polypeptide or an antigenic fragment thereof, a Neutrophil activating protein (NapA) polypeptide or an antigenic fragment thereof, a Decorin-binding protein A (DbpA) polypeptide or an antigenic fragment thereof, a Decorin-binding protein B (DbpB) polypeptide or an antigenic fragment thereof, an Outer surface protein C (OspC) polypeptide or an antigenic fragment thereof, an Outer surface protein A (OspA) polypeptide or an antigenic fragment thereof, an Outer surface protein B (OspB) polypeptide or an antigenic fragment thereof, a P100 polypeptide or an antigenic fragment thereof, a P41 polypeptide or an antigenic fragment thereof, a P66 polypeptide or an antigenic fragment thereof, a Borrelia membrane protein A (BmpA) polypeptide or an antigenic fragment thereof, a Borrelia membrane protein B (BmpB) polypeptide or an antigenic fragment thereof, a Borrelia membrane protein C (BmpC) polypeptide or an antigenic fragment thereof, a Borrelia glycosaminoglycan-binding protein (Bgp) polypeptide or an antigenic fragment thereof, and a Fibronectin-binding protein (Fbp) polypeptide or an antigenic fragment thereof.

11. The method of claim 10, wherein the one or more Lyme antigens are a mixture of a NapA polypeptide or an antigenic fragment thereof, a DbpA polypeptide or an antigenic fragment thereof, an OspC polypeptide or an antigenic fragment thereof, a P100 polypeptide or an antigenic fragment thereof, and a VlsE polypeptide or an antigenic fragment thereof.

12. The method of claim 10, wherein the one or more Lyme antigens are a mixture of an OspA polypeptide or an antigenic fragment thereof, a DbpA polypeptide or an antigenic fragment thereof, an OspC polypeptide or an antigenic fragment thereof, a P100 polypeptide or an antigenic fragment thereof, and a VlsE polypeptide or an antigenic fragment thereof.

13. The method of claim 10, wherein the one or more Lyme antigens are a mixture of a DbpA polypeptide or an antigenic fragment thereof, an OspC polypeptide or an antigenic fragment thereof, a P100 polypeptide or an antigenic fragment thereof, and a VlsE polypeptide or an antigenic fragment thereof.

14. The method of claim 10, wherein the one or more Lyme antigens are a mixture of an OspC polypeptide or an antigenic fragment thereof and a VlsE polypeptide or an antigenic fragment thereof.

15. The method of claim 10, wherein the one or more Lyme antigens are purified recombinant or synthetic polypeptides.

16. The method of claim 10, wherein the OspC polypeptide comprises an amino acid sequence having at least 80% sequence identity to any one of SEQ ID NOs:1-4, or to an antigenic fragment thereof.

17. The method of claim 10, wherein the P100 polypeptide comprises an amino acid sequence having at least 80% sequence identity to SEQ ID NO:5, or to an antigenic fragment thereof.

18. The method of claim 10, wherein the VlsE polypeptide comprises an amino acid sequence having at least 80% sequence identity to any one of SEQ ID NOs:6-7, or to an antigenic fragment thereof.

19. The method of claim 10, wherein the DbpA polypeptide comprises an amino acid sequence having at least 80% sequence identity to any one of SEQ ID NOs:8-10, or to an antigenic fragment thereof.

20. The method of claim 10, wherein the DbpB polypeptide comprises an amino acid sequence having at least 80% sequence identity to any one of SEQ ID NOs:11-13, or to an antigenic fragment thereof.

21. The method of claim 10, wherein the NapA polypeptide comprises an amino acid sequence having at least 80% sequence identity to any one of SEQ ID NOs:14-15, or to an antigenic fragment thereof.

22. The method of claim 10, wherein the OspA polypeptide comprises an amino acid sequence having at least 80% sequence identity to any one of SEQ ID NOs:16-18, or to an antigenic fragment thereof.

23. The method of claim 10, wherein the P41 polypeptide comprises an amino acid sequence having at least 80% sequence identity to any one of SEQ ID NOs:19-20, or to an antigenic fragment thereof.

24. The method of claim 10, wherein the BmpA polypeptide comprises an amino acid sequence having at least 80% sequence identity to any one of SEQ ID NOs:21-23, or to an antigenic fragment thereof.

25. The method of claim 1, further comprising observing in the subject a Lyme disease related symptom consisted of but not limited to a tick bite, erythema migrans, skin lesion, pain, fever, headache, and swelling.

26. The method of claim 1, further comprising measuring a Lyme antigen specific antibody in a blood sample of the subject using Western blot or enzyme-linked immunosorbent assay (ELISA).

27. A method for monitoring a treatment of Lyme disease in a subject, the method comprising:
a. providing a first sample of peripheral blood mononuclear cells (PBMCs) obtained from the subject before the treatment;
b. measuring a first level of one or more cytokines secreted by the first sample of PBMCs in response to stimulation by one or more Lyme antigens in serum-free medium;
c. providing a second sample of PBMCs obtained from the subject during or after a treatment of Lyme disease;
d. measuring a second level of one or more cytokines secreted by the second sample of PBMCs in response to stimulation by one or more Lyme antigens in serum-free medium; and
e. comparing the first level with the second level.

28. The method of claim 27, wherein the serum-free medium contains IL-7.

29. The method of claim 27, wherein the one or more cytokines are selected from the group consisting of IL-2, IL-3, IL-4, IL-5, IL-6, IL-9, IL-10, IL-13, IL-17A/F, IL-21, IL-22, IL-25, IL-31, TNF-α, TNF-β, IFN-γ, and GM-CSF.

30. The method of claim 27, wherein measuring one or more cytokines comprises performing a bioassay, an immunoassay, a flow cytometry, or a radioimmunoassay (RIA).

31. The method of claim 30, wherein the immunoassay is an enzyme-linked immunosorbent spot assay.

32. The method of claim 27, wherein the one or more Lyme antigens are selected from the group consisting of but not limited to a VlsE polypeptide or an antigenic fragment thereof, a NapA polypeptide or an antigenic fragment thereof, a DbpA polypeptide or an antigenic fragment thereof, a DbpB polypeptide or an antigenic fragment thereof, an OspC polypeptide or an antigenic fragment thereof, an OspA polypeptide or an antigenic fragment thereof, an OspB polypeptide or an antigenic fragment thereof, a P100 polypeptide or an antigenic fragment thereof, a P41 polypeptide or an antigenic fragment thereof, a P66 polypeptide or an antigenic fragment thereof, a BmpA polypeptide or an antigenic fragment thereof, a BmpB polypeptide or an antigenic fragment thereof, a BmpC polypeptide or an antigenic fragment thereof, a Bgp polypeptide or an antigenic fragment thereof, and a Fbp polypeptide or an antigenic fragment thereof.

33. The method of claim 32, wherein the one or more Lyme antigens are purified recombinant or synthetic polypeptide.

34. The method of claim 27, further comprising assessing the treatment of Lyme disease in the subject to be effective when the post-treatment level of one or more cytokines is lower than the baseline level.

35. The method of claim 27, further comprising assessing the treatment of Lyme disease in the subject to be ineffective when the post-treatment level of one or more cytokines is similar to or higher than the baseline level.

36. The method of claim 27, further comprising assessing in the subject a Lyme disease related symptom consisted of but not limited to a tick bite, erythema migrans, skin lesion, pain, fever, headache, and swelling.

37. The method of claim 27, further comprising measuring a Lyme antigen specific antibody in a blood sample of the subject using Western blot or ELISA before and after treatment.

38. A method of treating Lyme disease in a subject, the method comprising:
a. providing peripheral blood mononuclear cells (PBMCs) of the subject;
b. incubating the PBMCs in a serum-free medium with one or more Lyme antigens;
c. measuring the level of one or more cytokines secreted by the PBMCs;
d. if the level of the one or more cytokines is above a control level, administering to the subject a treatment comprising one or more antibiotics.

39. The method of claim 38, wherein the one or more antibiotics are selected from the group consisting of doxycycline, amoxicillin, cefuroxime axetil, ceftriaxone, cefotaxime, penicillin, and azithromycin.

40. The method of claim 38, wherein the serum-free medium contains interleukin-7.

41. The method of claim 38, wherein measuring the level of one or more cytokines comprises performing a bioassay, an immunoassay, a flow cytometry, or a radioimmunoassay (RIA).

42. The method of claim 41, wherein the immunoassay is an enzyme-linked immunosorbent spot (ELISpot) assay.

43. The method of claim 38, wherein the one or more cytokines are selected from the group consisting of IL-2, IL-3, IL-4, IL-5, IL-6, IL-9, IL-10, IL-13, IL-17A/F, IL-21, IL-22, IL-25, IL-31, TNF-α, TNF-β, IFN-γ, and GM-CSF.

44. The method of claim 43, wherein the cytokine is IFN-γ.

45. The method of claim 38, wherein the control level is the level of the one or more cytokines secreted by the peripheral blood mononuclear cells in a healthy subject.

46. The method of claim 38, wherein the one or more Lyme antigens are polypeptides or proteins derived from or exhibiting sequence similarity to polypeptides or proteins derived from one or more pathogenic species of Borrelia.

47. The method of claim 46, wherein the one or more pathogenic species of Borrelia is selected from the group consisting of Borrelia burgdorferi sensu stricto, Borrelia afzelii, Borrelia garinii, Borrelia valaisiana, Borrelia bissettii, Borrelia Lusitaniae, and Borrelia spielmanii.

48. The method of claim 46, wherein the one or more Lyme antigens are selected from the group consisting of but not limited to a Variable major protein-like gene E (VlsE) polypeptide or an antigenic fragment thereof, a Neutrophil activating protein (NapA) polypeptide or an antigenic fragment thereof, a Decorin-binding protein A (DbpA) polypeptide or an antigenic fragment thereof, a Decorin-binding protein B (DbpB) polypeptide or an antigenic fragment thereof, an Outer surface protein C (OspC) polypeptide or an antigenic fragment thereof, an Outer surface protein A (OspA) polypeptide or an antigenic fragment thereof, an Outer surface protein B (OspB) polypeptide or an antigenic fragment thereof, a P100 polypeptide or an antigenic fragment thereof, a P41 polypeptide or an antigenic fragment thereof, a P66 polypeptide or an antigenic fragment thereof, a Borrelia membrane protein A (BmpA) polypeptide or an antigenic fragment thereof, a Borrelia membrane protein B (BmpB) polypeptide or an antigenic fragment thereof, a Borrelia membrane protein C (BmpC) polypeptide or an antigenic fragment thereof, a Borrelia glycosaminoglycan-binding protein (Bgp) polypeptide or an antigenic fragment thereof, and a Fibronectin-binding protein (Fbp) polypeptide or an antigenic fragment thereof.

49. The method of claim 48, wherein the one or more Lyme antigens are a mixture of a NapA polypeptide or an antigenic fragment thereof, a DbpA polypeptide or an antigenic fragment thereof, an OspC polypeptide or an antigenic fragment thereof, a P100 polypeptide or an antigenic fragment thereof, and a VlsE polypeptide or an antigenic fragment thereof.

50. The method of claim 48, wherein the one or more Lyme antigens are a mixture of an OspA polypeptide or an antigenic fragment thereof, a DbpA polypeptide or an antigenic fragment thereof, an OspC polypeptide or an antigenic fragment thereof, a P100 polypeptide or an antigenic fragment thereof, and a VlsE polypeptide or an antigenic fragment thereof.

51. The method of claim 48, wherein the one or more Lyme antigens are a mixture of a DbpA polypeptide or an antigenic fragment thereof, an OspC polypeptide or an antigenic fragment thereof, a P100 polypeptide or an antigenic fragment thereof, and a VlsE polypeptide or an antigenic fragment thereof.

52. The method of claim 48, wherein the one or more Lyme antigens are a mixture of an OspC polypeptide or an antigenic fragment thereof and a VlsE polypeptide or an antigenic fragment thereof.

53. The method of claim 48, wherein the one or more Lyme antigens are purified recombinant or synthetic polypeptides.

54. The method of claim 48, wherein the OspC polypeptide comprises an amino acid sequence having at least 80% sequence identity to any one of SEQ ID NOs:1-4, or to an antigenic fragment thereof.

55. The method of claim 48, wherein the P100 polypeptide comprises an amino acid sequence having at least 80% sequence identity to SEQ ID NO:5, or to an antigenic fragment thereof.

56. The method of claim 48, wherein the VlsE polypeptide comprises an amino acid sequence having at least 80% sequence identity to any one of SEQ ID NOs:6-7, or to an antigenic fragment thereof.

57. The method of claim 48, wherein the DbpA polypeptide comprises an amino acid sequence having at least 80% sequence identity to any one of SEQ ID NOs:8-10, or to an antigenic fragment thereof.

58. The method of claim 48, wherein the DbpB polypeptide comprises an amino acid sequence having at least 80% sequence identity to any one of SEQ ID NOs:11-13, or to an antigenic fragment thereof.

59. The method of claim 48, wherein the NapA polypeptide comprises an amino acid sequence having at least 80% sequence identity to any one of SEQ ID NOs:14-15, or to an antigenic fragment thereof.

60. The method of claim 48, wherein the OspA polypeptide comprises an amino acid sequence having at least 80% sequence identity to any one of SEQ ID NOs:16-18, or to an antigenic fragment thereof.

61. The method of claim 48, wherein the P41 polypeptide comprises an amino acid sequence having at least 80% sequence identity to any one of SEQ ID NOs:19-20, or to an antigenic fragment thereof.

62. The method of claim 48, wherein the BmpA polypeptide comprises an amino acid sequence having at least 80% sequence identity to any one of SEQ ID NOs:21-23, or to an antigenic fragment thereof.

63. The method of claim 48, further comprising observing in the subject a Lyme disease related symptom consisted of but not limited to a tick bite, erythema migrans, skin lesion, pain, fever, headache, and swelling.

64. The method of claim 48, further comprising measuring a Lyme antigen specific antibody in a blood sample of the subject using Western blot or enzyme-linked immunosorbent assay (ELISA).

65. A composition comprising one or more Lyme antigen polypeptides selected from the group consisting of but not limited to a VlsE polypeptide or an antigenic fragment thereof, a NapA polypeptide or an antigenic fragment thereof, a DbpA polypeptide or an antigenic fragment thereof, a DbpB polypeptide or an antigenic fragment thereof, an OspC polypeptide or an antigenic fragment thereof, an OspA polypeptide or an antigenic fragment thereof, an OspB polypeptide or an antigenic fragment thereof, a P100 polypeptide or an antigenic fragment thereof, a P41 polypeptide or an antigenic fragment thereof, a P66 polypeptide or an antigenic fragment thereof, a BmpA polypeptide or an antigenic fragment thereof, a BmpB polypeptide or an antigenic fragment thereof, a BmpC polypeptide or an antigenic fragment thereof, a Bgp polypeptide or an antigenic fragment thereof, and a Fbp polypeptide or an antigenic fragment thereof.

66. The composition of claim 65, wherein the one or more Lyme antigen polypeptides are a mixture of a NapA polypeptide or an antigenic fragment thereof, a DbpA polypeptide or an antigenic fragment thereof, an OspC polypeptide or an antigenic fragment thereof, a P100 polypeptide or an antigenic fragment thereof, and a Vlse polypeptide or an antigenic fragment thereof.

67. The composition of claim 65, wherein the one or more Lyme antigen polypeptides are a mixture of an OspA polypeptide or an antigenic fragment thereof, a DbpA polypeptide or an antigenic fragment thereof, an OspC polypeptide or an antigenic fragment thereof, a P100 polypeptide or an antigenic fragment thereof, and a Vlse polypeptide or an antigenic fragment thereof.

68. The composition of claim 65, wherein the one or more Lyme antigen polypeptides are a mixture of a NapA polypeptide or an antigenic fragment thereof, a DbpA polypeptide or an antigenic fragment thereof, an OspC polypeptide or an antigenic fragment thereof, a P100 polypeptide or an antigenic fragment thereof, a Vlse polypeptide or an antigenic fragment thereof, and an OspA polypeptide or an antigenic fragment thereof.

69. The composition of claim 65, wherein the one or more Lyme antigen polypeptides are a mixture of a DbpA polypeptide or an antigenic fragment thereof, an OspC polypeptide or an antigenic fragment thereof, a P100 polypeptide or an antigenic fragment thereof, and a VlsE polypeptide or an antigenic fragment thereof.

70. The composition of claim 65, wherein the one or more Lyme antigen polypeptides are a mixture of an OspC polypeptide or an antigenic fragment thereof and a VlsE polypeptide or an antigenic fragment thereof.

71. The composition of claim 65, wherein the one or more Lyme antigen polypeptides are recombinant or synthetic polypeptide.

72. The composition of claim 65, wherein the OspC polypeptide comprises an amino acid sequence having at least 80% sequence identity to any one of SEQ ID NOs: 1-4, or to an antigenic fragment thereof.

73. The composition of claim 65, wherein the P100 polypeptide comprises an amino acid sequence having at least 80% sequence identity to SEQ ID NO:5, or to an antigenic fragment thereof.

74. The composition of claim 65, wherein the VlsE polypeptide comprises an amino acid sequence having at least 80% sequence identity to any one of SEQ ID NOs: 6-7, or to an antigenic fragment thereof.

75. The composition of claim 65, wherein the DbpA polypeptide comprises an amino acid sequence having at least 80% sequence identity to any one of SEQ ID NOs: 8-10, or to an antigenic fragment thereof.

76. The composition of claim 65, wherein the DbpB polypeptide comprises an amino acid sequence having at least 80% sequence identity to any one of SEQ ID NOs: 11-13, or to an antigenic fragment thereof.

77. The composition of claim 65, wherein the NapA polypeptide comprises an amino acid sequence having at least 80% sequence identity to any one of SEQ ID NOs: 14-15, or to an antigenic fragment thereof.

78. The composition of claim 65, wherein the OspA polypeptide comprises an amino acid sequence having at least 80% sequence identity to any one of SEQ ID NOs:16-18, or to an antigenic fragment thereof.

79. The composition of claim 65, wherein the P41 polypeptide comprises an amino acid sequence having at least 80% sequence identity to any one of SEQ ID NOs:19-20, or to an antigenic fragment thereof.

80. The composition of claim 65, wherein the BmpA polypeptide comprises an amino acid sequence having at least 80% sequence identity to any one of SEQ ID NOs:21-23, or to an antigenic fragment thereof.

81. A kit comprising the composition of claim 65.

82. The kit of claim 81, wherein the kit further comprises serum-free medium.

83. The kit of claim 81, wherein the kit further comprises interleukin-7.

84. The kit of claim 81, wherein the kit further comprises phytohaemagglutinin

85. The kit of claim 81, wherein the kit further comprises a solid phase support coated with one or more capture antibodies specific for a cytokine.

86. The kit of claim 81, wherein the cytokine is selected from the group consisting of but not limited to IL-2, IL-3, IL-4, IL-5, IL-6, IL-9, IL-10, IL-13, IL-17A/F, IL-21, IL-22, IL-25, IL-31, TNF-α, TNF-β, IFN-γ, and GM-CSF.

87. The kit of claim 86, wherein the cytokine is IFN-γ.

88. The kit of claim 81, wherein the solid phase support is a microwell of a microplate.

89. The kit of claim 81, wherein the kit further comprises a detection antibody specific for a cytokine.

90. The kit of claim 89, wherein the cytokine is selected from the group consisting of but not limited to IL-2, IL-3, IL-4, IL-5, IL-6, IL-9, IL-10, IL-13, IL-17A/F, IL-21, IL-22, IL-25, IL-31, TNF-α, TNF-β, IFN-γ, and GM-CSF.

91. The kit of claim 90, wherein the cytokine is IFN-γ.

92. The kit of claim 89, wherein the detection antibody is an enzyme-conjugated antibody.

93. The kit of claim 92, further comprising a chromogenic, fluorogenic, or electrochemiluminescent substrate of the enzyme.

94. The kit of claim 89, wherein the detection antibody is a biotinylated antibody.

95. The kit of claim 94, further comprising enzyme-conjugated streptavidin.

96. The kit of claim 95, further comprising a chromogenic, fluorogenic, or electrochemiluminescent substrate of the enzyme.

97. The kit of claim 89, wherein the detection antibody is an antibody tagged with a fluorescent dye.
